(19) United States
(12) Patent Application Publication
(10) Pub. No.: US 2011/0306573 A1
(43) Pub. Date: Dec. 15, 2011

(54) NUCLEOSIDE DERIVATIVES AS INHIBITORS OF VIRAL POLYMERASES

(76) Inventors: Salvatore Avolio, Rome (IT); Maria Emilia Di Francesco, Brookline, MA (US); Marco Pompei, Rome (IT); Vincenzo Summa, Rome (IT)

(21) Appl. No.: 13/061,746

(22) PCT Filed: Sep. 2, 2009
(86) PCT No.: PCT/EP2009/061330

§ 371 Date: Aug. 19, 2011

(30) Foreign Application Priority Data
Sep. 3, 2008 (GB) 0815968.3

(51) Int. Cl. A61K 31/7064; A61P 31/14; C07H 19/23; C12N 9/99; C07H 19/14

(52) U.S. Cl. 514/52; 435/184; 536/27.2; 536/26.26; 536/26.7; 514/43

(57) ABSTRACT

Compounds of structural formula (I): and pharmaceutically acceptable salts thereof; wherein R¹; R²; R³; Q¹ and Q² are as defined herein, processes for their preparation; pharmaceutical compositions containing them and their use in medicine, in particular the treatment or prevention of HCV infections, are disclosed.

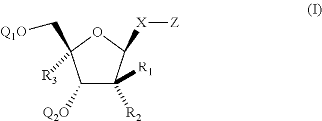

The present invention is concerned with nucleoside and nucleotide derivatives, their synthesis, and their use as inhibitors of RNA-dependent RNA viral polymerases. The compounds of the present invention are inhibitors of RNA-dependent RNA viral replication and are therefore useful for the treatment of RNA-dependent RNA viral infections. They are particularly useful as inhibitors of hepatitis C virus (HCV) NS5B polymerase, as inhibitors of HCV replication, and for the treatment of hepatitis C infection.

Hepatitis C virus (HCV) infection is a major health problem that leads to chronic liver disease, such as cirrhosis and hepatocellular carcinoma, in a substantial number of infected individuals, estimated to be 2-15% of the world's population. There are an estimated 4.5 million infected people in the United States alone, according to the U.S. Center for Disease Control. According to the World Health Organization, there are more than 200 million infected individuals worldwide, with at least 3 to 4 million people being infected each year. Once infected, about 20% of people clear the virus, but the rest harbor HCV the rest of their lives. Ten to twenty percent of chronically infected individuals eventually develop liver-destroying cirrhosis or cancer. The viral disease is transmitted parenterally by contaminated blood and blood products, contaminated needles, or sexually and vertically from infected mothers or carrier mothers to their off-spring. Current treatments for HCV infection, which are restricted to immunotherapy with recombinant interferon-α alone or in combination with the nucleoside analog ribavirin, are of limited clinical benefit. Moreover, there is no established vaccine for HCV. Consequently, there is an urgent need for improved therapeutic agents that effectively combat chronic HCV infection. Different approaches to HCV therapy have been taken, which include the inhibition of viral serine proteinase (NS3 protease), helicase, and RNA-dependent RNA polymerase (NS5B), and the development of a vaccine.

The HCV virion is an enveloped positive-strand RNA virus with a single oligoribonucleotide genomic sequence of about 9600 bases which encodes a polyprotein of about 3,010 amino acids. The protein products of the HCV gene consist of the structural proteins C, E1, and E2, and the non-structural proteins NS2, NS3, NS4A and NS4B, and NS5A and NS5B. The nonstructural (NS) proteins are believed to provide the catalytic machinery for viral replication. The NS3 protease releases NS5B, the RNA-dependent RNA polymerase from the polyprotein chain. HCV NS5B polymerase is required for the synthesis of a double-stranded RNA from a single-stranded viral RNA that serves as a template in the replication cycle of HCV. NS5B polymerase is therefore considered to be an essential component in the HCV replication complex [see K. Ishi, et al., “Expression of Hepatitis C Virus NS5B Protein: Characterization of Its RNA Polymerase Activity and RNA Binding,” Hepatology, 29: 1227-1235 (1999) and V. Lohmann, et al., “Biochemical and Kinetic Analyses of NS5B RNA-Dependent RNA Polymerase of the Hepatitis C Virus,” Virology, 249: 108-118 (1998)]. Inhibition of HCV NS5B polymerase prevents formation of the double-stranded HCV RNA and therefore constitutes an attractive approach to the development of HCV-specific antiviral therapies.

The development of inhibitors of HCV NS5B polymerase with potential for the treatment of HCV infection has been reviewed in M. P. Walker et al., “Promising candidates for the treatment of chronic hepatitis C,” Expert Opin. Invest. Drugs, 12: 1269-1280 (2003) and in P. Hoffmann et al., “Recent patents on experimental therapy for hepatitis C virus infection (1999-2002),” Expert Opin. Ther. Patents,” 13: 1707-1723 (2003). The activity of purine ribonucleosides against HCV polymerase was reported by A. E. Eldrup et al., “Structure-Activity Relationship of Purine Ribonucleosides for Inhibition of HCV RNA-Dependent RNA Polymerase,” J. Med. Chem., 47: 2283-2295 (2004). There is a continuing need for structurally diverse nucleoside derivatives as inhibitors of HCV polymerase as therapeutic approaches for HCV therapy.

The present invention provides a novel class of nucleosides and nucleotides that are potent inhibitors of RNA-dependent RNA viral replication and in particular HCV replication.

The present invention relates to compounds of structural formula (I):

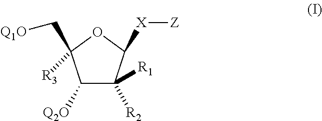

and pharmaceutically acceptable salts thereof; wherein:

X is an optionally substituted basic ring system found in nucleosides and nucleotide analogues X being linked to the carbohydrate ring through a N atom of the basic ring system;

Z is a 5 or 6 membered heterocyclic ring, containing one to three heteroatoms optionally substituted by an oxo, S(O)ₙ, S(O)ₙR⁴, C₁₋₄ alkyl, C₁₋₄ haloalkyl, CH₂OR⁴, CO₂R⁴, CONR⁴R⁵, NR⁴C(O)R⁵ or NR⁴R⁵ groups wherein R⁴ and R⁵ are independently selected from hydrogen and C₁₋₄ alkyl; and Z is attached to a ring atom of X that is two ring atoms from the N atom that links X to the carbohydrate ring;

R¹ is hydrogen, hydroxy, halo or C₁₋₆alkyl optionally substituted by fluoro;

R² is hydroxy, halo, OMe, C₁-C₁₆-alkylcarbonyl or hydrogen;

R³ is hydrogen or an azido, ethynyl, cyano or a C₁₋₆ aliphatic group optionally substituted by fluoro;

Q¹ is hydrogen or a mono-, di- or tri-phosphate group or a protecting group Q³ and

Q² is hydrogen or a protecting group Q⁴.

Suitably X is a purine, pyrrolopyrimidine, pyrazolopyrimidine or pyrimidine ring optionally substituted by halo, one or more oxo or hydroxy groups, or by one or more amino groups optionally substituted by COR⁶, wherein R⁶ is a C₁₋₆ aliphatic group or phenyl. Most suitably X is a pyrrolopyrimidine ring substituted by an amino group or a pyrazolopyrimidine ring substituted by an amino group. Preferably X is a pyrrolopyrimidine ring substituted at the 4-position by an amino group.

Suitably Z is a 5 or 6 membered heterocyclic ring, containing at least one heteroatom selected from oxygen, sulphur and nitrogen, and optionally substituted by an oxo, amino, or C₁₋₄ alkoxyl group, for example methoxy or a C₁₋₄ alkyl group, for example methyl. Suitably Z has a ring atom that is capable of hydrogen bonding to a hydrogen atom in a substituent on X. Most suitably Z is a 5 membered heterocyclic ring that contains two or three heteroatoms selected from oxygen and nitrogen, of which at most one is oxygen. Preferably Z is selected from 3-oxadiazole, 5-pyrazole and 2-oxazole.

Suitably R¹ is hydrogen, halo or C₁₋₄alkyl. More suitably R¹ is C₁₋₄alkyl or halo. More suitably R¹ is methyl or fluorine. Most suitably R¹ is methyl.

Suitably R² is hydroxy, hydrogen, chloro or fluoro. More suitably R² is hydroxy or halo. Most suitably R² is hydroxy or fluoro.

Suitably R³ is hydrogen, azido or methyl. More suitably R³ is hydrogen.

Suitable groups Q³ and Q⁴ are well known to those skilled in the art, for example those described in WO2006/065335 and PCT/EP2008/056128 which are incorporated herein by reference. For example Q³ may be C₁₋₁₆ alkylcarbonyl, C₂₋₁₈ alkenylcarbonyl, C₁₋₁₀ alkyloxycarbonyl, C₃₋₆ cycloalkylcarbonyl, C₃₋₆ cycloalkyloxycarbonyl or a monophosphate prodrug residue

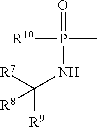

R⁷ is hydrogen, C₁₋₆alkyl optionally substituted with one substituent selected from the group consisting of fluoro, hydroxy, methoxy, amino, carboxy, carbamoyl, guanidino, mercapto, methylthio, 1H-imidazolyl, and 1H-indol-3-yl; or R⁷ is phenyl, benzyl or phenethyl each optionally substituted with one to two substituents independently selected from the group consisting of halogen, hydroxy, and methoxy;
R⁸ is hydrogen or methyl;
or R⁷ and R⁸ together with the carbon atom to which they attached form a 3- to 6-membered aliphatic spirocyclic ring system;
R⁹ is aryl, arylalkyl, heteroaryl or

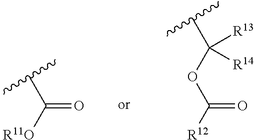

wherein R¹¹ is C₁₋₁₆alkyl, C₂₋₂₀alkenyl, (CH₂)₀₋₄C₇₋₉cycloalkyl, (CH₂)₀₋₄C₃₋₉cycloalkenyl or adamantly each optionally substituted with one to three substituents independently selected from halogen, hydroxy, carboxy, C₁₋₄alkoxy, trifluoromethyl and (CH₂)₀₋₄NR¹⁵R¹⁶ wherein R¹⁵ and R¹⁶ are independently selected from hydrogen and C₁₋₆alkyl; or R¹⁵ and R¹⁶, together with the nitrogen atom to which they are attached form a 4- to 7-membered heterocyclic ring optionally containing 1 or 2 more heteroatoms selected from N, O and S, which ring is optionally substituted by C₁₋₆ alkyl;
R¹⁰ is hydroxy or a group OR¹⁶ wherein R¹⁶ is CH₂OC(O)R¹⁷ or CH₂CH₂SR¹⁷ where R¹⁷ is C₁₋₆ alkylcarbonyl optionally substituted by a hydroxyl group or R¹⁶ is (CH₂)₂₋₄—O—(CH₂)₁₋₁₇CH₃, or an aromatic ring selected from phenyl, naphthyl, pyridinyl, pyrimidinyl, pyrazinyl, pyrazinyl, pyridazinyl, indolyl, quinolinyl, or isoquinolinyl, wherein the aromatic ring is optionally substituted with one to five substituents independently selected from the group consisting of halogen, C₁₋₄ alkyl, C₁₋₄ alkoxy, C₁₋₄ alkylthio, cyano, nitro, amino, carboxy, trifluoromethyl, trifluoromethoxy, C₁₋₄ alkylamino, di(C₁₋₄ alkyl)amino, C₁₋₄ alkylcarbonyl, C₁₋₄ alkylcarbonyloxy, and C₁₋₄ alkyloxycarbonyl; or R¹⁰ and Q⁴ form a bond to make a cyclic phosphate group;
R¹² is C₆₋₁₆alkyl, C₂₋₂₀alkenyl, (CH₂)₀₋₂C₇₋₉cycloalkyl, (CH₂)₀₋₂C₃₋₉cycloalkenyl, OC₁₋₆alkyl or adamantyl; and
R¹³ and R¹⁴ are independently selected from hydrogen and C₁₋₆alkyl;
or R¹³ and R¹⁴ together with the carbon atom to which they attached form a 3- to 6-membered aliphatic spirocyclic ring system;
and/or Q⁴ may be methyl, C₁₋₁₆ alkylcarbonyl, C₂₋₁₈ alkenylcarbonyl, C₁₋₁₀ alkyloxycarbonyl, C₃₋₆ cycloalkylcarbonyl, C₃₋₆ cycloalkyloxycarbonyl and an amino acyl residue of structural formula:

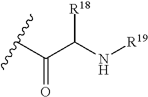

wherein R¹⁸ is hydrogen, C₁₋₅ alkyl or phenylC₀₋₂ alkyl; and R¹⁹ is hydrogen, C₁₋₄ alkyl, C₁₋₄ alkylsulfonyl or phenylC₀₋₂ alkylsulfonyl, or a group COR²⁰ wherein R²⁰ is C₁₋₄ alkyl optionally substituted by phenyl, C₁₋₄ alkoxy optionally substituted by phenyl, C₁₋₄alkylamino optionally substituted by C₁₋₄ alkyl optionally substituted by phenyl.

Suitably Q¹ is selected from hydrogen, monophosphate, diphosphate, or triphosphate, or C₁-C₁₆-alkylcarbonyl or a monophosphate prodrug of structure described before wherein: R⁷ is hydrogen, methyl or benzyl; more suitably hydrogen or methyl; R⁸ is hydrogen or methyl; more suitably hydrogen; R⁹ is Ph, CO₂R¹¹ or CR¹³R¹⁴OC(O)R¹² and R¹⁰ is hydroxyl or OR¹⁶; wherein R¹⁶ is an aromatic or heteroaromatic ring or CH₂CH₂SR¹⁷, where R¹⁷ is C₁-C₆ alkylcarbonyl, optionally substituted with a hydroxyl group; more suitably R¹⁰ is hydroxyl, O-phenyl or CH₂CH₂S—C₁-C₆-alkylcarbonyl optionally substituted with a hydroxyl group; most suitably R¹⁰ is hydroxyl or CH₂CH₂S S-tert-butylcarbonyl or CH₂CH₂S-hydroxy-tert-butylcarbonyl.

Suitably R¹¹ is C₁-C₁₆ alkyl, preferably C₇-C₁₆ alkyl; R¹² is C₁-C₁₆ alkyl, preferably C₇-C₁₆ alkyl; and R¹³ and R¹⁴ are both hydrogen.

Most suitably Q¹ is hydrogen or triphosphoryl.

Suitably Q² is selected from hydrogen, C₁-C₁₆-alkylcarbonyl or an amino acyl residue of the structure described before wherein R¹⁸ is hydrogen or C₁-C₅ alkyl, more suitably methyl, and R¹⁹ is hydrogen Most suitably Q² is hydrogen.

The compounds of formula (I) have the indicated stereochemical configuration.

Preferred embodiment the compound of the formula (I) include those compounds selected from the formula (II), (III), (IV) and (V):

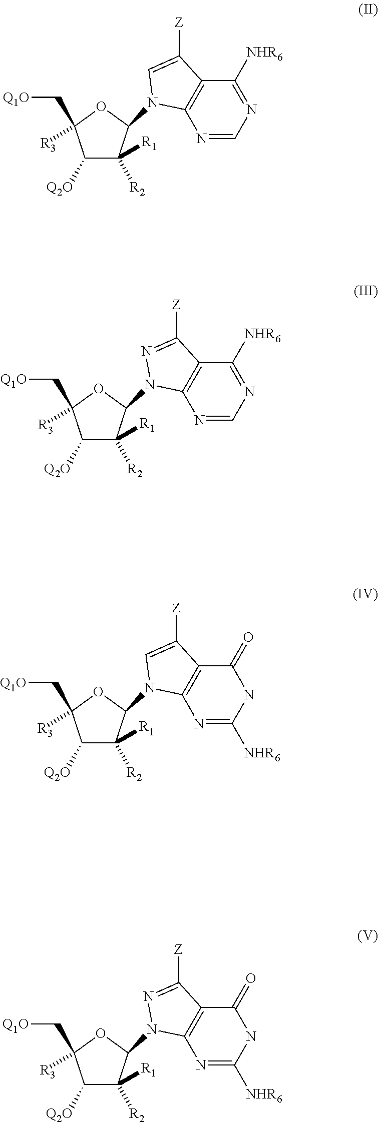

and pharmaceutically acceptable salts thereof; wherein R¹ to R⁶, Z, Q¹ and Q² are as hereinbefore defined.

Preferred compounds of the present invention include:
5-(2-methoxy-1,3-thiazol-4-yl)-7-(2-C-methyl-β-D-ribofuranosyl)-7H-pyrrolo[2,3-d]pyrimidin-4-amine,
7-(2-C-methyl-β-D-ribofuranosyl)-5-pyrimidin-2-yl-7H-pyrrolo[2,3-d]pyrimidin-4-amine,
7-(2-C-methyl-β-D-ribofuranosyl)-5-(1,3-oxazol-2-yl)-7H-pyrrolo[2,3-d]pyrimidin-4-amine,
7-(2-C-methyl-β-D-ribofuranosyl)-5-(1H-pyrazol-5-yl)-7H-pyrrolo[2,3-d]pyrimidin-4-amine,
7-(2-C-methyl-β-D-ribofuranosyl)-5-(1H-pyrazol-4-yl)-7H-pyrrolo[2,3-d]pyrimidin-4-amine,
7-(2-C-methyl-β-D-ribofuranosyl)-5-(1,2,4-oxadiazol-3-yl)-7H-pyrrolo[2,3-d]pyrimidin-4-amine,
7-(2-C-methyl-β-D-ribofuranosyl)-5-(1-methyl-1H-1,2,3-triazol-4-yl)-7H-pyrrolo[2,3-d]pyrimidin-4-amine,
7-(2-C-methyl-β-D-ribofuranosyl)-5-(2-thienyl)-7H-pyrrolo[2,3-d]pyrimidin-4-amine,
7-(2-C-methyl-5-triphospho-β-D-ribofuranosyl)-5-(1-methyl-1H-tetrazol-5-yl)-7H-pyrrolo[2,3-d]pyrimidin-4-amine,
7-(2-C-methyl-5-triphospho-β-D-ribofuranosyl)-5-(2-methyl-2H-tetrazol-5-yl)-7H-pyrrolo[2,3-d]pyrimidin-4-amine,
7-(2-C-methyl-5-triphospho-β-D-ribofuranosyl)-5-(1,3-oxazol-2-yl)-7H-pyrrolo[2,3-d]pyrimidin-4-amine,
5-(2-methoxy-1,3-thiazol-4-yl)-7-(2-C-methyl-5-triphospho-β-D-ribofuranosyl)-7H-pyrrolo[2,3-d]pyrimidin-4-amine,
7-(2-C-methyl-5-triphospho-β-D-ribofuranosyl)-5-(1H-pyrazol-5-yl)-7H-pyrrolo[2,3-d]pyrimidin-4-amine,
7-(2-C-methyl-5-triphospho-β-D-ribofuranosyl)-5-(1H-pyrazol-4-yl)-7H-pyrrolo[2,3-d]pyrimidin-4-amine,
7-(2-C-methyl-5-triphospho-β-D-ribofuranosyl)-5-(1,2,4-oxadiazol-3-yl)-7H-pyrrolo[2,3-d]pyrimidin-4-amine,
7-(2-C-methyl-5-triphospho-β-D-ribofuranosyl)-5-(1-methyl-1H-1,2,3-triazol-4-yl)-7H-pyrrolo[2,3-d]pyrimidin-4-amine,
7-(2-deoxy-2-fluoro-2-methyl-5-triphospho-β-D-ribofuranosyl)-5-(1H-pyrazol-5-yl)-7H-pyrrolo[2,3-d]pyrimidin-4-amine,
7-(2-deoxy-2-fluoro-2-methyl-5-triphospho-β-D-ribofuranosyl)-5-(1,3-oxazol-2-yl)-7H-pyrrolo[2,3-d]pyrimidin-4-amine,
7-(2-C-methyl-5-triphospho-β-D-ribofuranosyl)-5-pyrimidin-2-yl-7H-pyrrolo[2,3-d]pyrimidin-4-amine,
7-(2-deoxy-2-fluoro-2-methyl-5-triphospho-β-D-ribofuranosyl)-5-(1,2,4-oxadiazol-3-yl)-7H-pyrrolo[2,3-d]pyrimidin-4-amine,
7-(2-deoxy-2-fluoro-2-methyl-β-D-ribofuranosyl)-5-(1H-pyrazol-5-yl)-7H-pyrrolo[2,3-d]pyrimidin-4-amine,
7-(2-C-methyl-β-D-ribofuranosyl)-5-(6-methoxypyridin-3-yl)-7H-pyrrolo[2,3-d]pyrimidin-4-amine,
7-(2-deoxy-2-fluoro-2-methyl-β-D-ribofuranosyl)-5-(1,2,4-oxadiazol-3-yl)-7H-pyrrolo[2,3-d]pyrimidin-4-amine,
7-(2-deoxy-2-fluoro-2-methyl-β-D-ribofuranosyl)-5-(1,3-oxazol-2-yl)-7H-pyrrolo[2,3-d]pyrimidin-4-amine,
1-(2-C-methyl-β-D-ribofuranosyl)-3-(1H-pyrazol-3-yl)-1H-pyrazolo[3,4-d]pyrimidin-4-amine,
1-[5-O-(hydroxy{[hydroxy(phosphonooxy)phosphoryl]oxy}phosphoryl)-2-C-methyl- -D-ribofuranosyl]-3-(1H-pyrazol-3-yl)-1H-pyrazolo[3,4-d]pyrimidin-4-amine,
7-(2-C-methyl-β-D-ribofuranosyl)-5-(1,3,4-oxadiazol-2-yl)-7H-pyrrolo[2,3-d]pyrimidin-4-amine,
7-(2-C-methyl-5-triphospho-β-D-ribofuranosyl)-5-(1,3,4-oxadiazol-2-yl)-7H-pyrrolo[2,3-d]pyrimidin-4-amine,
Ethyl 2-{[(R)-({(2R,3S,4R,5R)-5-[4-amino-5-(1H-pyrazol-5-yl)-7H-pyrrolo[2,3-d]pyrimidin-7-yl]-3,4-dihydroxy-4-methyltetrahydrofuran-2-yl}methoxy)(phenoxy)phosphoryl]amino}propanoate,
Ethyl 2-{[(R)-({(2R,3S,4R,5R)-5-[4-amino-5-(1,3-oxazol-2-yl)-7H-pyrrolo[2,3-d]pyrimidin-7-yl]-3,4-dihydroxy-4-methyltetrahydrofuran-2-yl}methoxy)(phenoxy)phosphoryl]amino}propanoate
   and pharmaceutically acceptable salts thereof.

The compounds of formula (I) are useful as inhibitors of RNA-dependent RNA viral polymerases and in particular of HCV NS5B polymerase. They are also inhibitors of RNA-dependent RNA viral replication and in particular of HCV replication and are useful for the treatment of RNA-dependent RNA viral infections and in particular for the treatment of HCV infection. The compounds of the formula (I) wherein Q¹ and Q² are other than 5′-triphosphate and hydroxyl respectively may act as prodrugs or may be converted into compounds of the formula (I) which are useful for the treatment of RNA-dependent RNA viral infection and in particular for the treatment of HCV infection.

Without limitation as to their mechanism of action, prodrugs of the compounds of the present invention as herein defined act as precursors of the corresponding nucleoside 5′-monophosphates. Endogenous kinase enzymes convert the 5′-monophosphates into their 5′-triphosphate derivatives which are the inhibitors of the RNA-dependent RNA viral polymerases. Thus, the prodrugs may provide for more efficient target cell penetration than the nucleoside itself, may be less susceptible to metabolic degradation, and may have the ability to target a specific tissue, such as the liver, resulting in a wider therapeutic index allowing for lowering the overall dose of the antiviral agent.

Also encompassed within the present invention are pharmaceutical compositions containing the compounds alone or in combination with other agents active against RNA-dependent RNA viruses and in particular against HCV as well as methods for the inhibition of RNA-dependent RNA viral replication and for the treatment of RNA-dependent RNA viral infections.

In one embodiment of the present invention, the compounds of the present invention are useful as precursors to inhibitors of positive-sense single-stranded RNA-dependent RNA viral polymerases, inhibitors of positive-sense single-stranded RNA-dependent RNA viral replication, and/or for the treatment of positive-sense single-stranded RNA-dependent RNA viral infections. In a class of this embodiment, the positive-sense single-stranded RNA-dependent RNA virus is a Flaviviridae virus or a Picornaviridae virus. In a subclass of this class, the Picornaviridae virus is a rhinovirus, a poliovirus, or a hepatitis A virus. In a second subclass of this class, the Flaviviridae virus is selected from the group consisting of hepatitis C virus, yellow fever virus, dengue virus, West Nile virus, Japanese encephalitis virus, Banzi virus, and bovine viral diarrhea virus (BVDV). In a subclass of this subclass, the Flaviviridae virus is hepatitis C virus.

Another aspect of the present invention is concerned with a method for inhibiting RNA-dependent RNA viral polymerases, a method for inhibiting RNA-dependent RNA viral replication, and/or a method for treating RNA-dependent RNA viral infections in a mammal in need thereof comprising administering to the mammal a therapeutically effective amount of a compound of structural formula (I).

In one embodiment of this aspect of the present invention, the RNA-dependent RNA viral polymerase is a positive-sense single-stranded RNA-dependent RNA viral polymerase. In a class of this embodiment, the positive-sense single-stranded RNA-dependent RNA viral polymerase is a Flaviviridae viral polymerase or a Picornaviridae viral polymerase. In a subclass of this class, the Picornaviridae viral polymerase is rhinovirus polymerase, poliovirus polymerase, or hepatitis A virus polymerase. In a second subclass of this class, the Flaviviridae viral polymerase is selected from the group consisting of hepatitis C virus polymerase, yellow fever virus polymerase, dengue virus polymerase, West Nile virus polymerase, Japanese encephalitis virus polymerase, Banzi virus polymerase, and bovine viral diarrhea virus (BVDV) polymerase. In a subclass of this subclass, the Flaviviridae viral polymerase is hepatitis C virus polymerase.

In a second embodiment of this aspect of the present invention, the RNA-dependent RNA viral replication is a positive-sense single-stranded RNA-dependent RNA viral replication, such as a Flaviviridae viral replication or Picornaviridae viral replication. In one subclass, the Picornaviridae viral replication is rhinovirus replication, poliovirus replication, or hepatitis A virus replication. In a second subclass, the Flaviviridae viral replication is selected from the group consisting of hepatitis C virus replication, yellow fever virus replication, dengue virus replication, West Nile virus replication, Japanese encephalitis virus replication, Banzi virus replication, and bovine viral diarrhea virus replication and preferably hepatitis C virus replication.

In a third embodiment of this aspect of the present invention, the RNA-dependent RNA viral infection is a positive-sense single-stranded RNA-dependent viral infection such as a Flaviviridae viral infection or Picornaviridae viral infection. In a subclass of this class, the Picornaviridae viral infection is rhinovirus infection, poliovirus infection, or hepatitis A virus infection. In a second subclass of this class, the Flaviviridae viral infection is selected from the group consisting of hepatitis C virus infection, yellow fever virus infection, dengue virus infection, West Nile virus infection, Japanese encephalitis virus infection, Banzi virus infection, and bovine viral diarrhea virus infection Preferably, the Flaviviridae viral infection is hepatitis C virus infection.

Throughout the instant application, the following terms have the indicated meanings:

The alkyl groups specified above are intended to include those alkyl groups of the designated length in either a straight or branched configuration. Exemplary of such alkyl groups are methyl, ethyl, propyl, isopropyl, butyl, sec-butyl, tertiary butyl, pentyl, isopentyl, hexyl, isohexyl, heptyl, 1-propylbutyl, octyl, 2-propylpentyl, and the like.

The term “adamantyl” encompasses both 1-adamantyl and 2-adamantyl.

The term “alkenyl” shall mean straight or branched chain alkenes of two to twenty total carbon atoms, or any number within this range (e.g., ethenyl, propenyl, butenyl, pentenyl, oleyl, etc.).

The term “cycloalkyl” shall mean cyclic rings of alkanes having the designated number of carbon atoms, or any number within this range (examples include cyclopropyl, cyclobutyl, cyclopentyl, cyclohexyl, cycloheptyl, or cyclooctyl).

The term “cycloalkenyl” shall mean cyclic rings of alkenes having the designated number of carbon atoms, or any number within this range (i.e., cyclopropenyl, cyclobutenyl, cycloheptenyl, cyclohexenyl, cycloheptenyl, or cyclooctenyl).

The term “C₁₋₆ aliphatic group” refers to alkyl, alkenyl, alkynyl, cycloalkyl, cycloalkenyl or cycloalkynyl groups that contain from one to six carbon atoms.

The term “alkoxy” refers to straight or branched chain alkoxides of the number of carbon atoms specified (e.g., C₁₋₄alkoxy), or any number within this range [i.e., methoxy, ethoxy, isopropoxy, etc.].

The term “alkylamino” refers to straight or branched alkylamines of the number of carbon atoms specified (e.g., C₁₋₄alkylamino), or any number within this range [i.e., methylamino, ethylamino, isopropylamino, t-butylamino, etc.].

The term “alkylsulfonyl” refers to straight or branched chain alkylsulfones of the number of carbon atoms specified (e.g., C₁₋₆alkylsulfonyl), or any number within this range [i.e., methylsulfonyl (MeSO₂—), ethylsulfonyl, isopropylsulfonyl, etc.].

The term “alkyloxycarbonyl” refers to straight or branched chain esters of a carboxylic acid or carbamic acid group present in a compound of the present invention having the number of carbon atoms specified (e.g., C₁₋₈alkyloxycarbonyl), or any number within this range [i.e., methyloxycarbonyl (MeOCO—), ethyloxycarbonyl, or butyloxycarbonyl].

The term “alkylcarbonyl” refers to straight or branched chain alkyl acyl group of the specified number of carbon atoms (e.g., C₁₋₈alkylcarbonyl), or any number within this range [i.e., methyloxycarbonyl (MeOCO—), ethyloxycarbonyl, or butyloxycarbonyl].

The term “halo” is intended to include fluoro, chloro, bromo and iodo [i.e. chloro or fluoro].

The term “monophosphate” refers to —P(O)(OH)₂, The term “diphosphate” refers to the radical having the structure:

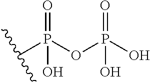

and the term “triphosphate” refers to the radical having the structure:

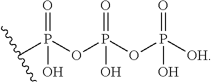

The term “substituted” shall be deemed to include multiple degrees of substitution by a named substituent. Where multiple substituent moieties are disclosed or claimed, the substituted compound can be independently substituted by one or more of the disclosed or claimed substituent moieties, singly or plurally.

The term “5′-triphosphate” refers to a triphosphoric acid ester derivative of the 5′-hydroxyl group of a nucleoside compound of the present invention having the following general structural formula:

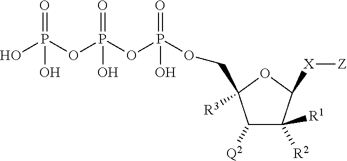

wherein R¹, R², R³, X, Q² and Z are as defined above.

The term “composition”, as in “pharmaceutical composition,” is intended to encompass a product comprising the active ingredient(s) and the inert ingredient(s) that make up the carrier, as well as any product which results, directly or indirectly, from combination, complexation or aggregation of any two or more of the ingredients, or from dissociation of one or more of the ingredients, or from other types of reactions or interactions of one or more of the ingredients. Accordingly, the pharmaceutical compositions of the present invention encompass any composition made by admixing a compound of the present invention and a pharmaceutically acceptable carrier.

The terms “administration of” and “administering a” compound should be understood to mean providing a compound of the invention or a prodrug of a compound of the invention to the individual in need.

Another aspect of the present invention is concerned with a method of inhibiting HCV NS5B polymerase, inhibiting HCV replication, or treating HCV infection with a compound of the present invention in combination with one or more agents useful for treating HCV infection. Such agents active against HCV include, but are not limited to, ribavirin, levovirin, viramidine, nitazoxanide, thymosin alpha-1, interferon-β, interferon-α, pegylated interferon-α (peginterferon-α), a combination of interferon-α and ribavirin, a combination of peginterferon-α and ribavirin, a combination of interferon-α and levovirin, and a combination of peginterferon-α and levovirin. Interferon-α includes, but is not limited to, recombinant interferon-α2a (such as Roferon interferon available from Hoffmann-LaRoche, Nutley, N.J.), pegylated interferon-α2a (Pegasys™) interferon-α2b (such as Intron-A interferon available from Schering Corp., Kenilworth, N.J.), pegylated interferon-α2b (PegIntron™), a recombinant consensus interferon (such as interferon alphacon-1), and a purified interferon-α product. Amgen's recombinant consensus interferon has the brand name Infergen®. Levovirin is the L-enantiomer of ribavirin which has shown immunomodulatory activity similar to ribavirin. Viramidine represents an analog of ribavirin disclosed in WO 01/60379 (assigned to ICN Pharmaceuticals). In accordance with this method of the present invention, the individual components of the combination can be administered separately at different times during the course of therapy or concurrently in divided or single combination forms. The instant invention is therefore to be understood as embracing all such regimes of simultaneous or alternating treatment, and the term “administering” is to be interpreted accordingly. It will be understood that the scope of combinations of the compounds of this invention with other agents useful for treating HCV infection includes in principle any combination with any pharmaceutical composition for treating HCV infection. When a compound of the present invention or a pharmaceutically acceptable salt thereof is used in combination with a second therapeutic agent active against HCV, the dose of each compound may be either the same as or different from the dose when the compound is used alone.

For the treatment of HCV infection, the compounds of the present invention may also be administered in combination with an agent that is an inhibitor of HCV NS3 serine protease. HCV NS3 serine protease is an essential viral enzyme and has been described to be an excellent target for inhibition of HCV replication. Both substrate and non-substrate based inhibitors of HCV NS3 protease inhibitors are disclosed in WO 98/22496, WO 98/46630, WO 99/07733, WO 99/07734, WO 99/38888, WO 99/50230, WO 99/64442, WO 00/09543, WO 00/59929, GB-2337262, WO 02/18369, WO 02/08244, WO 02/48116, WO 02/48172, WO 05/037214, and U.S. Pat. No. 6,323,180. HCV NS3 protease as a target for the development of inhibitors of HCV replication and for the treatment of HCV infection is discussed in B. W. Dymock, “Emerging therapies for hepatitis C virus infection,” Emerging Drugs, 6: 13-42 (2001). Specific HCV NS3 protease inhibitors combinable with the compounds of the present invention include BILN2061, VX-950, SCH6, SCH7, and SCH-503034.

Ribavirin, levovirin, and viramidine may exert their anti-HCV effects by modulating intracellular pools of guanine nucleotides via inhibition of the intracellular enzyme inosine monophosphate dehydrogenase (IMPDH). IMPDH is the rate-limiting enzyme on the biosynthetic route in de novo guanine nucleotide biosynthesis. Ribavirin is readily phosphorylated intracellularly and the monophosphate derivative is an inhibitor of IMPDH. Thus, inhibition of IMPDH represents another useful target for the discovery of inhibitors of HCV replication. Therefore, the compounds of the present invention may also be administered in combination with an inhibitor of IMPDH, such as VX-497, which is disclosed in WO 97/41211 and WO 01/00622 (assigned to Vertex); another IMPDH inhibitor, such as that disclosed in WO 00/25780 (assigned to Bristol-Myers Squibb); or mycophenolate mofetil [see A. C. Allison and E. M. Eugui, Agents Action, 44 (Suppl.): 165 (1993)].

For the treatment of HCV infection, the compounds of the present invention may also be administered in combination with the antiviral agent amantadine (1-aminoadamantane) [for a comprehensive description of this agent, see J. Kirschbaum, Anal. Profiles Drug Subs. 12: 1-36 (1983)].

The compounds of the present invention may also be combined for the treatment of HCV infection with antiviral 2′-C-branched ribonucleosides disclosed in R. E. Harry-O'kuru, et al., J. Org. Chem., 62: 1754-1759 (1997); M. S. Wolfe, et al., Tetrahedron Lett., 36: 7611-7614 (1995); U.S. Pat. No. 3,480,613 (Nov. 25, 1969); U.S. Pat. No. 6,777,395 (Aug. 17, 2004); U.S. Pat. No. 6,914,054 (Jul. 5, 2005); International Publication Numbers WO 01/90121 (29 Nov. 2001); WO 01/92282 (6 Dec. 2001); WO 02/32920 (25 Apr. 2002); WO 02/057287 (25 Jul. 2002); WO 02/057425 (25 Jul. 2002); WO 04/002422 (8 Jan. 2004); WO 04/002999 (8 Jan. 2004); WO 04/003000 (8 Jan. 2004); WO 04/002422 (8 Jan. 2004); US Patent Application Publications 2005/0107312; US 2005/0090463; US 2004/0147464; and US 2004/0063658; the contents of each of which are incorporated by reference in their entirety. Such 2′-C-branched ribonucleosides include, but are not limited to, 2′-C-methylcytidine, 2′-fluoro-2′-C-methylcytidine 2′-C-methyluridine, 2′-C-methyladenosine, 2′-C-methylguanosine, and 9-(2-C-methyl-β-D-ribofuranosyl)-2,6-diaminopurine; the corresponding amino acid esters of the furanose C-2′, C-3′, and C-5′ hydroxyls (such as 3′-O-(L-valyl)-2′-C-methylcytidine dihydrochloride, also referred to as valopicitabine dihydrochloride or NM-283 and 3′-O-(L-valyl)-2′-fluoro-2′-C-methylcytidine), and the corresponding optionally substituted cyclic 1,3-propanediol esters of their 5′-phosphate derivatives.

The compounds of the present invention may also be combined for the treatment of HCV infection with other nucleosides having anti-HCV properties, such as those disclosed in U.S. Pat. No. 6,864,244 (Mar. 8, 2005); WO 02/51425 (4 Jul. 2002), assigned to Mitsubishi Pharma Corp.; WO 01/79246, WO 02/32920, and WO 02/48165 (20 Jun. 2002), assigned to Pharmasset, Ltd.; WO 01/68663 (20 Sep. 2001), assigned to ICN Pharmaceuticals; WO 99/43691 (2 Sep. 1999); WO 02/18404 (7 Mar. 2002), assigned to Hoffmann-LaRoche; U.S. 2002/0019363 (14 Feb. 2002); WO 02/100415 (19 Dec. 2002); WO 03/026589 (3 Apr. 2003); WO 03/026675 (3 Apr. 2003); WO 03/093290 (13 Nov. 2003): US 2003/0236216 (25 Dec. 2003); US 2004/0006007 (8 Jan. 2004); WO 04/011478 (5 Feb. 2004); WO 04/013300 (12 Feb. 2004); US 2004/0063658 (1 Apr. 2004); and WO 04/028481 (8 Apr. 2004).

In one embodiment, nucleoside HCV NS5B polymerase inhibitors that may be combined with the nucleoside derivatives of the present invention are selected from the following compounds: 4′-azido-cytidine; 4-amino-7-(2-C-methyl-β-D-ribofuranosyl)-7H-pyrrolo[2,3-d]pyrimidine; 4-amino-7-(2-C-hydroxymethyl-β-D-ribofuranosyl)-7H-pyrrolo[2,3-d]pyrimidine; 4-amino-7-(2-C-fluoromethyl-β-D-ribofuranosyl)-7H-pyrrolo[2,3-d]pyrimidine; 4-amino-5-fluoro-7-(2-C-methyl-β-D-ribofuranosyl)-7H-pyrrolo[2,3-d]pyrimidine; 2-amino-7-(2-C-methyl-β-D-ribofuranosyl)-7H-pyrrolo[2,3-d]pyrimidin-4(3H)-one; 4-amino-7-(2-C,2-O-dimethyl-β-D-ribofuranosyl)-7H-pyrrolo[2,3-d]pyrimidine; β-D-2′-deoxy-2′-fluoro-2′-C-methyl -cytidine and pharmaceutically acceptable salts and prodrugs thereof.

The compounds of the present invention may also be combined for the treatment of HCV infection with non-nucleoside inhibitors of HCV polymerase such as those disclosed in WO 01/77091 (18 Oct. 2001), assigned to Tularik, Inc.; WO 01/47883 (5 Jul. 2001), assigned to Japan Tobacco, Inc.; WO 02/04425 (17 Jan. 2002), assigned to Boehringer Ingelheim; WO 02/06246 (24 Jan. 2002), assigned to Istituto di Ricerche di Biologia Molecolare P. Angeletti S.p.A.; WO 02/20497 (3 Mar. 2002); WO 2005/016927 (in particular JTK003), assigned to Japan Tobacco, Inc.; the contents of each of which are incorporated herein by reference in their entirety; and HCV-796 (Viropharma Inc.).

In one embodiment, non-nucleoside HCV NS5B polymerase inhibitors that may be combined with the nucleoside derivatives of the present invention are selected from the following compounds: 14-cyclohexyl-6-[2-(dimethylamino)ethyl]-7-oxo-5,6,7,8-tetrahydroindolo[2,1-a][2,5]benzodiazocine-11-carboxylic acid; 14-cyclohexyl-6-(2-morpholin-4-ylethyl)-5,6,7,8-tetrahydroindolo[2,1-a][2,5]benzodiazocine-11-carboxylic acid; 14-cyclohexyl-6-[2-(dimethylamino)ethyl]-3-methoxy-5,6,7,8-tetrahydroindolo[2,1-a][2,5]benzodiazocine-11-carboxylic acid; 14-cyclohexyl-3-methoxy-6-methyl-5,6,7,8-tetrahydroindolo[2,1-a][2,5]benzodiazocine-11-carboxylic acid; methyl({[(14-cyclohexyl-3-methoxy-6-methyl-5,6,7,8-tetrahydroindolo[2,1-a][2,5]benzodiazocin-11-yl)carbonyl]amino}sulfonyl)acetate; ({[(14-cyclohexyl-3-methoxy-6-methyl-5,6,7,8-tetrahydroindolo[2,1-a][2,5]benzodiazocin-11-yl)carbonyl]amino}sulfonyl)acetic acid; 14-cyclohexyl-N-[(dimethylamino)sulfonyl]-3-methoxy-6-methyl-5,6,7,8-tetrahydroindolo[2,1-a][2,5]benzodiazocine-11-carboxamide; 3-chloro-14-cyclohexyl-6-[2-(dimethylamino)ethyl]-7-oxo-5,6,7,8-tetrahydroindolo[2,1-a][2,5]benzodiazocine 11-carboxylic acid; N′-(11-carboxy-14-cyclohexyl-7,8-dihydro-6H-indolo[1,2-e][1,5]benzoxazocin-7-yl)-N,N-dimethylethane-1,2-diaminium bis(trifluoroacetate); 14-cyclohexyl-7,8-dihydro-6H-indolo[1,2-e][1,5]benzoxazocine-11-carboxylic acid; 14-cyclohexyl-6-methyl-7-oxo-5,6,7,8-tetrahydroindolo[2,1-a][2,5]benzodiazocine-11-carboxylic acid; 14-cyclohexyl-3-methoxy-6-methyl-7-oxo-5,6,7,8-tetrahydroindolo[2,1-a][2,5]benzodiazocine-11-carboxylic acid; 14-cyclohexyl-6-[2-(dimethylamino)ethyl]-3-methoxy-7-oxo-5,6,7,8-tetrahydroindolo[2,1-a][2,5]benzodiazocine-11-carboxylic acid; 14-cyclohexyl-6-[3-(dimethylamino)propyl]-7-oxo-5,6,7,8-tetrahydroindolo[2,1-a][2,5]benzodiazocine-11-carboxylic acid; 14-cyclohexyl-7-oxo-6-(2-piperidin-1-ylethyl)-5,6,7,8-tetrahydroindolo[2,1-a][2,5]benzodiazocine-11-carboxylic acid; 14-cyclohexyl-6-(2-morpholin-4-ylethyl)-7-oxo-5,6,7,8-tetrahydroindolo[2,1-a][2,5]benzodiazocine-11-carboxylic acid; 14-cyclohexyl-6-[2-(diethylamino)ethyl]-7-oxo-5,6,7,8-tetrahydroindolo[2,1-a][2,5]benzodiazocine-11-carboxylic acid; 14-cyclohexyl-6-(1-methylpiperidin-4-yl)-7-oxo-5,6,7,8-tetrahydroindolo[2,1-a][2,5]benzodiazocine-11-carboxylic acid; 14-cyclohexyl-N-[(dimethylamino)sulfonyl]-7-oxo-6-(2-piperidin-1-ylethyl)-5,6,7,8-tetrahydroindolo[2,1-a][2,5]benzodiazocine-11-carboxamide; 14-cyclohexyl-6-[2-(dimethylamino)ethyl]-N-[(dimethylamino)sulfonyl]-7-oxo-5,6,7,8-tetrahydroindolo[2,1-a][2,5]benzodiazocine-11-carboxamide; 14-cyclopentyl-6-[2-(dimethylamino)ethyl]-7-oxo-5,6,7,8-tetrahydroindolo[2,1-a][2,5]benzodiazocine-11-carboxylic acid; 14-cyclohexyl-5,6,7,8-tetrahydroindolo[2,1-a][2,5]benzodiazocine-11-carboxylic acid; 6-allyl-14-cyclohexyl-3-methoxy-5,6,7,8-tetrahydroindolo[2,1-a][2,5]benzodiazocine-11-carboxylic acid; 14-cyclopentyl-6-[2-(dimethylamino)ethyl]-5,6,7,8-tetrahydroindolo[2,1-a][2,5]benzodiazocine-11-carboxylic acid; 14-cyclohexyl-6-[2-(dimethylamino)ethyl]-5,6,7,8-tetrahydroindolo[2,1-a][2,5]benzodiazocine-11-carboxylic acid; 13-cyclohexyl-5-methyl-4,5,6,7-tetrahydrofuro[3′,2′:6,7][1,4]diazocino[1,8-a]indole-10-carboxylic acid; 15-cyclohexyl-6-[2-(dimethylamino)ethyl]-7-oxo-6,7,8,9-tetrahydro-5H-indolo[2,1-a][2,6]benzodiazonine-12-carboxylic acid; 15-cyclohexyl-8-oxo-6,7,8,9-tetrahydro-5H-indolo[2,1-a][2,5]benzodiazonine-12-carboxylic acid; 13-cyclohexyl-6-oxo-6,7-dihydro-5H-indolo[1,2-d][1,4]benzodiazepine-10-carboxylic acid; and pharmaceutically acceptable salts thereof.

By “pharmaceutically acceptable” is meant that the carrier, diluent, or excipient must be compatible with the other ingredients of the formulation and not deleterious to the recipient thereof.

Also included within the present invention are pharmaceutical compositions comprising the compounds of the present invention in association with a pharmaceutically acceptable carrier. Another example of the invention is a pharmaceutical composition made by combining any of the compounds described above and a pharmaceutically acceptable carrier. Another illustration of the invention is a process for making a pharmaceutical composition comprising combining any of the compounds described above and a pharmaceutically acceptable carrier.

Also included within the present invention are pharmaceutical compositions useful for inhibiting RNA-dependent RNA viral polymerases in particular HCV NS5B polymerase comprising an effective amount of a compound of the present invention and a pharmaceutically acceptable carrier. Pharmaceutical compositions useful for treating RNA-dependent RNA viral infections in particular HCV infection are also encompassed by the present invention as well as a method of inhibiting RNA-dependent RNA viral polymerases in particular HCV NS5B polymerase and a method of treating RNA-dependent viral replication and in particular HCV replication. Additionally, the present invention is directed to a pharmaceutical composition comprising a therapeutically effective amount of a compound of the present invention in combination with a therapeutically effective amount of another agent active against RNA-dependent RNA viruses and in particular against HCV. Agents active against HCV include, but are not limited to, ribavirin, levovirin, viramidine, thymosin alpha-1, an inhibitor of HCV NS3 serine protease, interferon-α, pegylated interferon-α (peginterferon-α), a combination of interferon-α and ribavirin, a combination of peginterferon-α and ribavirin, a combination of interferon-α and levovirin, and a combination of peginterferon-α and levovirin. Interferon-α includes, but is not limited to, recombinant interferon-α2a (such as Roferon interferon available from Hoffmann-LaRoche, Nutley, N.J.), interferon-α2b (such as Intron-A interferon available from Schering Corp., Kenilworth, N.J.), a consensus interferon, and a purified interferon-α product. For a discussion of ribavirin and its activity against HCV, see J. O, Saunders and S. A. Raybuck, “Inosine Monophosphate Dehydrogenase: Consideration of Structure, Kinetics, and Therapeutic Potential,” Ann. Rep. Med. Chem., 35: 201-210 (2000).

Another aspect of the present invention provides for the use of the compounds of the present invention and their pharmaceutical compositions for the manufacture of a medicament for the inhibition of RNA-dependent RNA viral replication, in particular HCV replication, and/or the treatment of RNA-dependent RNA viral infections, in particular HCV infection. Yet a further aspect of the present invention provides for the compounds of the present invention and their pharmaceutical compositions for use as a medicament for the inhibition of RNA-dependent RNA viral replication, in particular HCV replication, and/or for the treatment of RNA-dependent RNA viral infections, in particular HCV infection.

The pharmaceutical compositions of the present invention comprise a compound of formula (I) as an active ingredient or a pharmaceutically acceptable salt thereof, and may also contain a pharmaceutically acceptable carrier and optionally other therapeutic ingredients.

The compositions include compositions suitable for oral, rectal, topical, parenteral (including subcutaneous, intramuscular, and intravenous), ocular (ophthalmic), pulmonary (nasal or buccal inhalation), or nasal administration, although the most suitable route in any given case will depend on the nature and severity of the conditions being treated and on the nature of the active ingredient. They may be conveniently presented in unit dosage form and prepared by any of the methods well-known in the art of pharmacy.

In practical use, the compounds of formula (I) can be combined as the active ingredient in intimate admixture with a pharmaceutical carrier according to conventional pharmaceutical compounding techniques. The carrier may take a wide variety of forms depending on the form of preparation desired for administration, e.g., oral or parenteral (including intravenous). In preparing the compositions for oral dosage form, any of the usual pharmaceutical media may be employed, such as, for example, water, glycols, oils, alcohols, flavoring agents, preservatives, coloring agents and the like in the case of oral liquid preparations, such as, for example, suspensions, elixirs and solutions; or carriers such as starches, sugars, microcrystalline cellulose, diluents, granulating agents, lubricants, binders, disintegrating agents and the like in the case of oral solid preparations such as, for example, powders, hard and soft capsules and tablets, with the solid oral preparations being preferred over the liquid preparations.

Because of their ease of administration, tablets and capsules represent the most advantageous oral dosage unit form in which case solid pharmaceutical carriers are obviously employed. If desired, tablets may be coated by standard aqueous or nonaqueous techniques. Such compositions and preparations should contain at least 0.1 percent of active compound. The percentage of active compound in these compositions may, of course, be varied and may conveniently be between about 2 percent to about 60 percent of the weight of the unit. The amount of active compound in such therapeutically useful compositions is such that an effective dosage will be obtained. The active compounds can also be administered intranasally as, for example, liquid drops or spray.

The tablets, pills, capsules, and the like may also contain a binder such as gum tragacanth, acacia, corn starch or gelatin; excipients such as dicalcium phosphate; a disintegrating agent such as corn starch, potato starch, alginic acid; a lubricant such as magnesium stearate; and a sweetening agent such as sucrose, lactose or saccharin. When a dosage unit form is a capsule, it may contain, in addition to materials of the above type, a liquid carrier such as a fatty oil.

Various other materials may be present as coatings or to modify the physical form of the dosage unit. For instance, tablets may be coated with shellac, sugar or both. A syrup or elixir may contain, in addition to the active ingredient, sucrose as a sweetening agent, methyl and propylparabens as preservatives, a dye and a flavoring such as cherry or orange flavor.

Compounds of formula I may also be administered parenterally. Solutions or suspensions of these active compounds can be prepared in water suitably mixed with a surfactant such as hydroxy-propylcellulose. Dispersions can also be prepared in glycerol, liquid polyethylene glycols and mixtures thereof in oils. Under ordinary conditions of storage and use, these preparations contain a preservative to prevent the growth of microorganisms.

The pharmaceutical forms suitable for injectable use include sterile aqueous solutions or dispersions and sterile powders for the extemporaneous preparation of sterile injectable solutions or dispersions. In all cases, the form must be sterile and must be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (e.g. glycerol, propylene glycol and liquid polyethylene glycol), suitable mixtures thereof, and vegetable oils.

Any suitable route of administration may be employed for providing a mammal, especially a human with an effective dosage of a compound of the present invention. For example, oral, rectal, topical, parenteral, ocular, pulmonary, nasal, and the like may be employed. Dosage forms include tablets, troches, dispersions, suspensions, solutions, capsules, creams, ointments, aerosols, and the like. Preferably, compounds of structural formula I are administered orally. Also preferably, compounds of structural formula I are administered parenterally.

For oral administration to humans, the dosage range is 0.01 to 1000 mg/kg body weight in divided doses. In one embodiment the dosage range is 0.1 to 100 mg/kg body weight in divided doses. In another embodiment the dosage range is 0.5 to 20 mg/kg body weight in divided doses. For oral administration, the compositions are preferably provided in the form of tablets or capsules containing 1.0 to 1000 milligrams of the active ingredient, particularly, 1, 5, 10, 15, 20, 25, 50, 75, 100, 150, 200, 250, 300, 400, 500, 600, 750, 800, 900, and 1000 milligrams of the active ingredient for the symptomatic adjustment of the dosage to the patient to be treated.

The effective dosage of active ingredient employed may vary depending on the particular compound employed, the mode of administration, the condition being treated and the severity of the condition being treated. Such dosage may be ascertained readily by a person skilled in the art. This dosage regimen may be adjusted to provide the optimal therapeutic response.

The compounds of the present invention contain one or more asymmetric centers and can thus occur as racemates and racemic mixtures, single enantiomers, diastereoisomeric mixtures and individual diastereoisomers. When R¹⁸ in the amino acyl residue embodiment of Q² is a substituent other than hydrogen in the formula

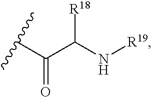

the amino acyl residue contains an asymmetric center and is intended to include the individual R- and S-stereoisomers as well as RS-diastereoisomeric mixtures. In one embodiment, the stereochemistry at the stereogenic carbon corresponds to that of an S-amino acid, that is, the naturally occurring alpha-amino acid stereochemistry, as depicted in the formula:

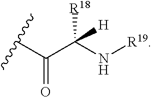

Furthermore, when R⁹ is:

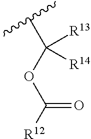

and R¹³ and R¹⁴ are not both hydrogen, the carboxy residue contains an asymmetric center and is intended to include the individual R- and S-stereoisomers as well as RS-stereoisomeric mixtures. Thus, when R⁴ and R⁵ are also not both hydrogen, the aminoalcohol residue contains two asymmetric centers and is intended to include the individual R,R-, R,S-, S,R- and S,S-diastereoisomers as well as mixtures thereof.

The present invention is meant to comprehend compounds having the β-D stereochemical configuration for the five-membered furanose ring as depicted in the structural formula, that is, nucleoside phosphoramidates in which the substituents at C-1 and C-4 of the five-membered furanose ring have the β-stereochemical configuration (“up” orientation as denoted by a bold line). Some of the compounds described herein contain olefinic double bonds, and unless specified otherwise, are meant to include both E and Z geometric isomers.

Some of the compounds described herein may exist as tautomers such as keto-enol tautomers. The individual tautomers as well as mixtures thereof are encompassed with compounds of structural formula (I).

Compounds of structural formula (I) may be separated into their individual diastereoisomers by, for example, fractional crystallization from a suitable solvent, for example methanol or ethyl acetate or a mixture thereof, or via chiral chromatography using an optically active stationary phase.

Alternatively, any stereoisomer of a compound of the structural formula (I) may be obtained by stereospecific synthesis using optically pure starting materials or reagents of known configuration.

The compounds of the present invention may be administered in the form of a pharmaceutically acceptable salt. The term “pharmaceutically acceptable salt” refers to salts prepared from pharmaceutically acceptable non-toxic bases or acids including inorganic or organic bases and inorganic or organic acids. Salts of basic compounds encompassed within the term “pharmaceutically acceptable salt” refer to non-toxic salts of the compounds of this invention which are generally prepared by reacting the free base with a suitable organic or inorganic acid. Representative salts of basic compounds of the present invention include, but are not limited to, the following: acetate, benzenesulfonate, benzoate, bicarbonate, bisulfate, bitartrate, borate, bromide, camsylate, carbonate, chloride, clavulanate, citrate, dihydrochloride, edetate, edisylate, estolate, esylate, fumarate, gluceptate, gluconate, glutamate, glycollylarsanilate, hexylresorcinate, hydrabamine, hydrobromide, hydrochloride, hydroxynaphthoate, iodide, isothionate, lactate, lactobionate, laurate, malate, maleate, mandelate, mesylate, methylbromide, methylnitrate, methylsulfate, mucate, napsylate, nitrate, N-methylglucamine ammonium salt, oleate, oxalate, pamoate (embonate), palmitate, pantothenate, phosphate/diphosphate, polygalacturonate, salicylate, stearate, sulfate, subacetate, succinate, tannate, tartrate, teoclate, tosylate, triethiodide and valerate. Furthermore, where the compounds of the invention carry an acidic moiety, suitable pharmaceutically acceptable salts thereof include, but are not limited to, salts derived from inorganic bases including aluminum, ammonium, calcium, copper, ferric, ferrous, lithium, magnesium, manganic, mangamous, potassium, sodium, zinc, and the like. Particularly preferred are the ammonium, calcium, magnesium, potassium, and sodium salts. Salts derived from pharmaceutically acceptable organic non-toxic bases include salts of primary, secondary, and tertiary amines, cyclic amines, and basic ion-exchange resins, such as arginine, betaine, caffeine, choline, N,N-dibenzylethylenediamine, diethylamine, 2-diethylaminoethanol, 2-dimethylaminoethanol, ethanolamine, ethylenediamine, N-ethylmorpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, tromethamine, and the like.

Also, in the case of a carboxylic acid (—COOH) or hydroxyl group being present in the compounds of the present invention, pharmaceutically acceptable prodrug esters of carboxylic acid derivatives, such as methyl, ethyl, or pivaloyloxymethyl esters or prodrug acyl derivatives of the ribose C-2′, C-3′, and C-5′ hydroxyls, such as O-acetyl, O-pivaloyl, O-benzoyl and O-aminoacyl, can be employed. Included are those esters and acyl groups known in the art for modifying the bioavailability, tissue distribution, solubility, and hydrolysis characteristics for use as sustained-release or prodrug formulations. The contemplated derivatives are readily convertible in vivo into the required compound. Thus, in the methods of treatment of the present invention, the terms “administering” and “administration” is meant to encompass the treatment of the viral infections described with a compound specifically disclosed or with a compound which may not be specifically disclosed, but which converts to the specified compound in vivo after administration to the mammal, including a human patient. Conventional procedures for the selection and preparation of suitable prodrug derivatives are described, for example, in “Design of Prodrugs,” ed. H. Bundgaard, Elsevier, 1985, which is incorporated by reference herein in its entirety.

The compounds of the present invention may be prepared by the general methods outlined in the following Schemes. Nucleoside analogues of structure 1-2 (Scheme 1) wherein W is carbon or nitrogen and R¹ to R³, Z, Q¹ and Q² are as hereinbefore defined and R^{a} and R^{b} are substituents for X as hereinbefore defined, can be obtained via metal-mediated cross coupling reactions between a functionalized and optionally protected nucleoside derivative such as 1-1 and a suitable heterocyclic derivative. A variety of reaction partners can be employed, including derivatives in which A is halogen, (preferably bromine or iodine), trialkyltin, boronic acid and boronic esters and B is hydrogen, halogen, trialkyltin, boronic acid and boronic esters. The nucleoside component can be optionally protected with suitable oxygen and nitrogen protecting groups by employing established synthetic methodologies (see for example Greene, T. W. and Wuts, P. G. M, Protective Groups in Organic Synthesis, Wiley-Interscience). Reactions are carried out in the presence of a suitable metal catalyst/ligand including Pd(PPh₃)₄, PdCl₂(PPh₃)₂, CuI/trans-1,2-diaminecyclohexane or others known to those skilled in the art; a suitable non-nucleophilic base might also be employed (for example trialkylamine or sodium carbonate or cesium carbonate or others). Coupling reactions can be performed in solvents such as dimethylformamide and dioxane, at temperatures in the range of 80-120° C., with or without the use of microwave irradiation. Further functionalisation of the O- and N-moieties might be required after cross-coupling and optional deprotection steps.

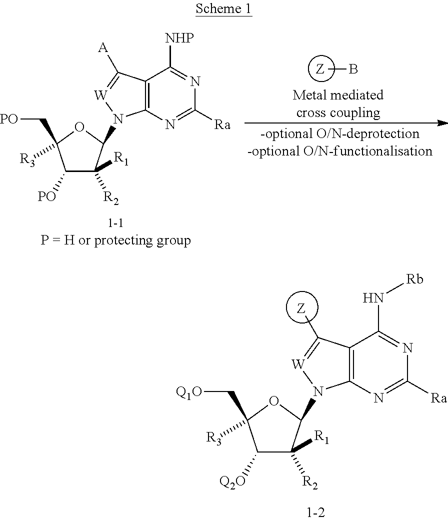

In a similar manner, compounds of structure 2-3 and 2-5 (Scheme 2) wherein R¹ to R³, Z, Q¹ and Q² are as hereinbefore defined and R^{b} is a substituent for X as hereinbefore defined, can be prepared via metal mediated cross coupling reaction of an appropriate heterocyclic component with a suitably functionalized cytidine derivative 2-2 or uridine derivative 2-4. In turn, intermediate 2-2 can be accessed either from an optionally protected cytidine derivative 2-1 or from a functionalized, optionally protected uridine derivative 2-4, via carbonyl activation and reaction with an amine derivative (preferably NH₃) according to established synthetic methods (Chemistry of Nucleosides and Nucleotides, Vol. 1, 2, 3, edited by Townsend, Plenum press). Furthermore, compounds of structure 2-3 can be obtained also from functionalized, optionally protected uridine derivatives 2-5 via carbonyl activation, reaction with an amine derivative (preferably NH₃) and optional deprotection. Further functionalisation of the O- and N-moieties might be required after cross-coupling and optional deprotection steps.

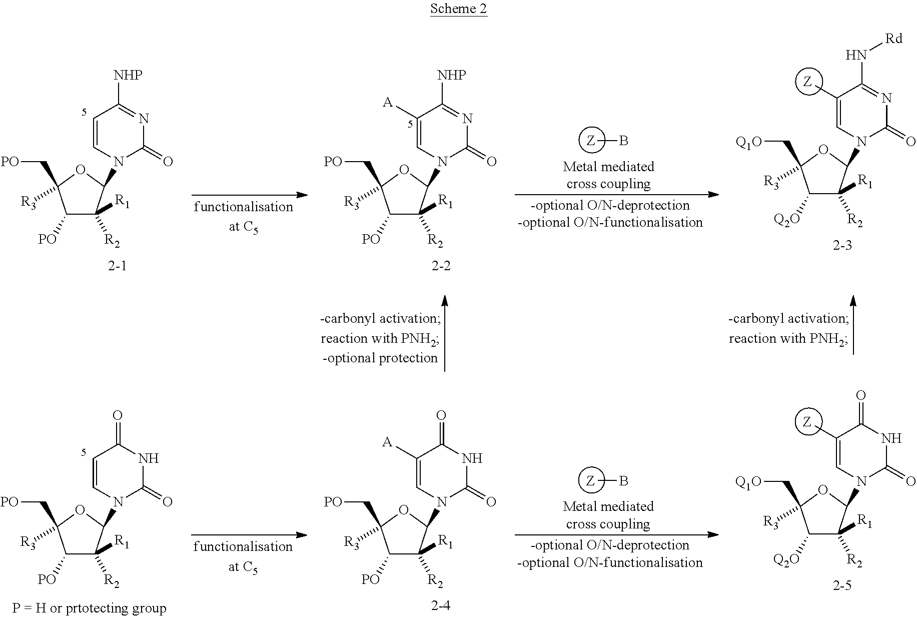

Some of the compounds of this invention can also be prepared as described in Scheme 3. A nucleoside derivative of structure 3-1, wherein W is carbon or nitrogen and R¹ to R³, Z, Q¹ and Q² are as hereinbefore defined and R^{a} and R^{b} are substituents for X as hereinbefore defined and where E can be nitrogen, CH, C-Alk or C—Ar, can be reacted with an appropriate organic azide (R′=trialkylsilylmethyl, trialkyltin) to give, after further functionalisation (for example: nitrogen alkylation), optional O/N-deprotection and further optional O/N-functionalisation compounds 3-2 and 3-3.

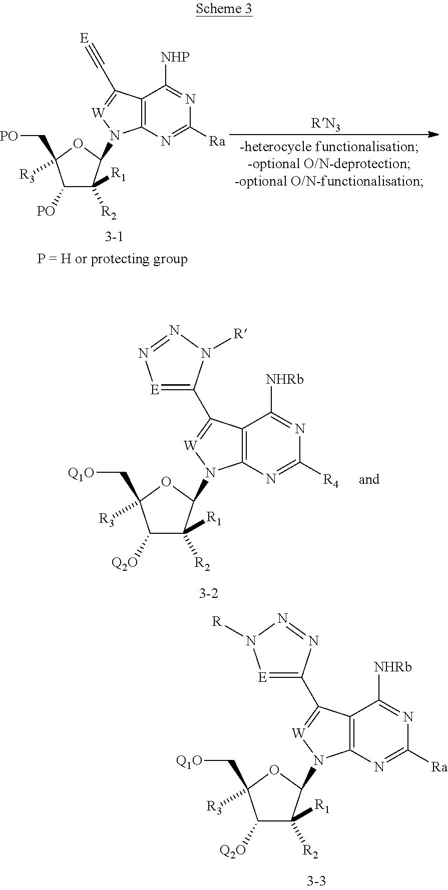

A further way to prepare the compounds described herein is depicted in Scheme 4.
An optionally protected nucleoside derivative of structure 4-1 wherein W is carbon or nitrogen and R¹ to R³, Q¹ and Q² are as hereinbefore defined and R^{a} and R^{b} are substituents for X as hereinbefore defined and R^{d} is a substuent for Z as hereinbefore defined, can be reacted with hydroxylamine followed by treatment with an orthoester or a carboxylic acid derivative to give, after optional deprotection, products of structure 4-2.

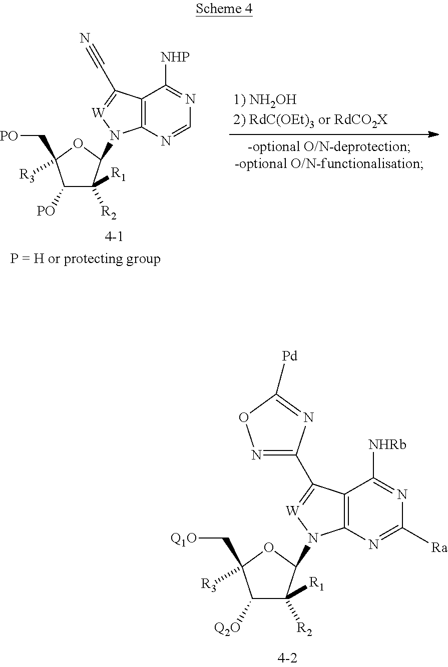

The nucleoside analogues herein described wherein W is carbon or nitrogen and R¹ to R³, Z, Q¹ and Q² are as hereinbefore defined, can be converted into their corresponding monophophates, diphosphates and triphosphates employing known methods, as described in Scheme 5 for one of the structural classes of the compounds of this invention.

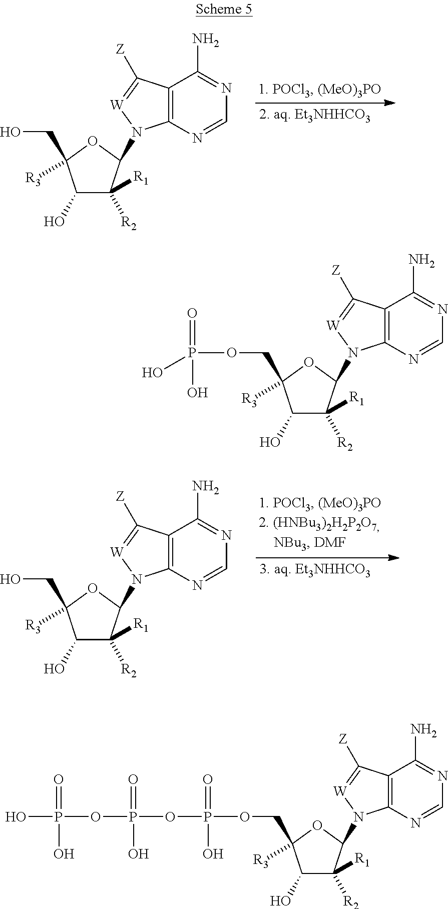

Further compounds of the present invention can also be prepared as described in Scheme 6. An optionally protected nucleoside derivative of structure 6-1 can be converted into a 1,3,4-oxadiazole nucleoside derivative of structure 6-4, wherein W is carbon or nitrogen, R¹ to R³, Q1 and Q2, R^{b} and R^{d} are as hereinbefore defined. In particular 6-1 can be converted to the corresponding carboxylic acid with one of the methods known to those skilled in art (e.g. by treatment with sodium chlorite in the presence t-butanol, 2-methyl-2-butene and sodium phosphate monobasic) and further progressed to a hydrazide derivative of structure 6-2 (e.g.: by coupling of the previously obtained carboxylic acid with tert-butyl hydrazinecarboxylate and subsequent removal of the N-Boc protecting group). Finally, treatment with an orthoester in the presence of a Lewis acid (e.g.: CH(OEt)₃, BF₃.Et₂O) followed by optional deprotection and functional group manipulation can give the required oxadiazole derivative of structure 6-4.

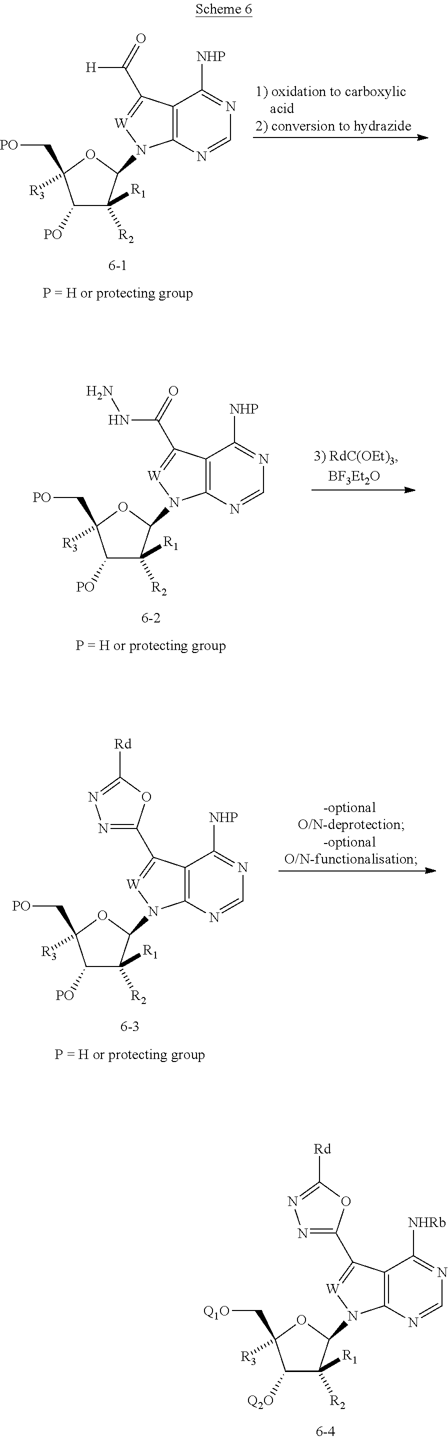

The nucleoside analogues herein described can also be converted into their corresponding monophosphate prodrugs as described in Scheme 7 employing methods known to those skilled in the art (e.g.: Uchiyama, M. et al. J. Org. Chem., 1993, 373; Kamaike, K. et al. Nucleosides & Nucleotides, 1987, 6, 699; Nishida, A. et al. J. Org. Chem., 1988, 53, 3386). In particular a phosphoroamidate prodrug of structure 7-3, wherein W is carbon or nitrogen, Z, R¹ to R³ and R⁷ to R¹⁰ are as hereinbefore defined can be obtained from an optionally protected nucleoside derivative of formula 7-1 (e.g.: P=tetrhydropyranyl group) by treatment with tert-butylmagnesium chloride followed by a suitable phosphorochloridate reagent of structure 7-2 (e.g.: McGuigan, C. et al. J. Med. Chem., 2005, 48, 3504). Removal of the optional protecting group can be necessary to obtain the required derivatives 7-3 (e.g. acid treatment with 80% aq. formic acid)

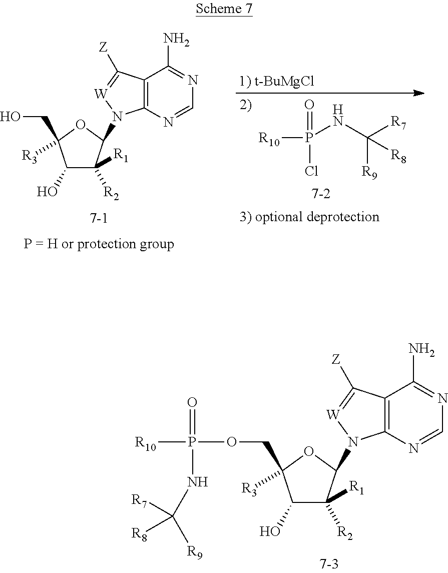

### General Synthetic Procedures

All solvents were obtained from commercial sources and were used without further purification. With the exception of routine deprotection and coupling steps, reactions were carried out under an atmosphere of nitrogen in oven dried (110° C.) glassware. Organic extracts were dried over sodium sulfate, and were concentrated (after filtration of the drying agent) on rotary evaporators operating under reduced pressure. Flash chromatography was carried out either on silica gel following published procedure (W. C. Still et al., J. Org. Chem. 1978, 43, 2923) or on semi-automated flash chromatography systems utilizing pre-packed columns.

Reagents were usually obtained directly from commercial suppliers (and used as supplied) but a limited number of compounds from in-house corporate collections were utilised. In the latter case the reagents are readily accessible using routine synthetic steps that are either reported in the scientific literature or are known to those skilled in the art.

¹H, ¹⁹F and ³¹P nmr spectra were recorded on Bruker AM series spectrometers operating at (reported) frequencies between 300 and 600 MHz. Chemical shifts (δ) for signals corresponding to non-exchangeable protons (and exchangeable protons where visible) are recorded in parts per million (ppm) relative to tetramethylsilane and are measured using the residual solvent peak as reference. Signals are tabulated in the order: multiplicity (s, singlet; d, doublet; t, triplet; q, quartet; m, multiplet; br, broad, and combinations thereof); coupling constant(s) in hertz; number of protons. Mass spectral (MS) data were obtained on Waters Micromass ZMD, operating in negative (ES⁻) or positive (ES⁺) ionization mode and results are reported as the ratio of mass over charge (m/z). Preparative scale HPLC separations were carried out on: 1) Waters Delta Prep 4000 preparative chromatography system, equipped with a Waters 2487 Dual λ absorbance detector; 2) Automated (UV-triggered) RP-HPLC Shimadzu Discovery VP system, incorporating an LC-8A preparative liquid chromatography module, an SPD-10A UV-VIS detector and a FRC-10A fraction collector module. In both cases the stationary phase employed was an Atlantis Prep T3 5 μm OBD (19×150 mm) or a XBridge Prep C₁₈ 5 μm OBD (19×150 mm). Unless otherwise stated, the mobile phase comprised a linear gradient of binary mixture of MeCN (containing 0.1% TFA) and water (containing 0.1% TFA), or MeCN and 5 mM dimethylhexylammonium bicarbonate in water using flow rates between 15 and 25 mL/min. Reactions under microwave irradiation were carried out in Emrys Optimizer reactor from Personal Chemistry, Sweden.

The following abbreviations are used in the Schemes and Examples: AcOH: acetic acid; aq.: aqueous; bs: broad singlet; bt: broad triplet; DBU: 1,8-Diazabicyclo[5.4.0]undec-7-ene; DIAD: diisopropyl azodicarboxylate; DIPEA: diisopropylethyl amine; DMF: dimethylformamide; DMSO: dimethylsulfoxide; eq.: equivalent(s); Et₂O: diethyl ether; EtOAc: ethyl acetate; EtOH: ethanol; (HNBu₃)₂H₂P₂O₇: bis tributylammonium pyrophosphate; h: hour(s); M: molar; MeCN: acetonitrile; MeOH: methanol; (MeO)₃PO: trimethyl phosphate; min: minutes; NaBH₃CN: sodium cyanoborohydride; NBu₃: tributylamine; NMP: 1-methyl-2-pyrrolidinone; Pd (PPh₃)₄: tetrakis(triphenylphosphine)palladium (0); PE: petroleum ether; P(O)Cl₃: phosphorous oxychloride; RP-HPLC: reversed phase high-performance liquid chromatography; RT: room temperature; SPE: solid phase extraction; TBDMS: tert-butyldimethylsilyl; TEA: triethylamine; TFA: trifluoroacetic acid; THF: tetrahydrofuran.

### Triphosphate Synthesis: General Procedure

Neat POCl₃ (2.5 eq) was added dropwise via a syringe to a 0.15 M solution the appropriate nucleoside (previously dried by coevaporation with pyridine and toluene) in trimethyl phosphate (stored over sieves) at 0° C. or RT. After stirring the resulting mixture for 2 h at 0° C. or at RT, a 0.5 M solution of bis tributylammonium pyrophosphate (6.0 eq) and tributylamine (5.0 eq) in DMF was added in one portion to the reaction mixture under vigorous stirring. After vigorous stirring for 1 min at 0° C. or RT, triethylammonium hydrogenocarbonate buffer (1 M aq., 50 eq, pH=7.5) was added to the reaction mixture, which was then stirred for further 3 h at RT and concentrated under reduced pressure (cold bath). The residue was dissolved in water and the triphosphate was recovered by anion exchange SPE with a buffer system of 0.5 M TEAB (pH=7.5) followed by RP-HPLC purification (mobile phase: 5 mM dimethylhexylammonium bicarbonate, pH=8.0/MeCN) to afford the title compounds as tris dimethylhexylammonium salts (oils). Typical yields ranged from 2% to 40%.

### REPRESENTATIVE EXAMPLES

The compounds of the present invention were also evaluated for cellular toxicity and anti-viral specificity in the counterscreens described below.

While the invention has been described and illustrated in reference to specific embodiments thereof, those skilled in the art will appreciate that various changes, modifications, and substitutions can be made therein without departing from the spirit and scope of the invention. For example, effective dosages other than the preferred doses as set forth hereinabove may be applicable as a consequence of variations in the responsiveness of the human being treated for severity of the HCV infection. Likewise, the pharmacologic response observed may vary according to and depending upon the particular active compound selected or whether there are present pharmaceutical carriers, as well as the type of formulation and mode of administration employed, and such expected variations or differences in the results are contemplated in accordance with the objects and practices of the present invention. It is intended therefore that the invention be limited only by the scope of the claims which follow and that such claims be interpreted as broadly as is reasonable.

### Example 1

### Entry 1, Table 1

### 7-(2-C-methyl-β-D-ribofuranosyl)-5-(1-methyl-1H-tetrazol-5-yl)-7H-pyrrolo[2,3-d]pyrimidin-4-amine (A) and 7-(2-C-methyl-β-D-ribofuranosyl)-5-(2-methyl-2H-tetrazol-5-yl)-7H-pyrrolo[2,3-d]pyrimidin-4-amine (B)

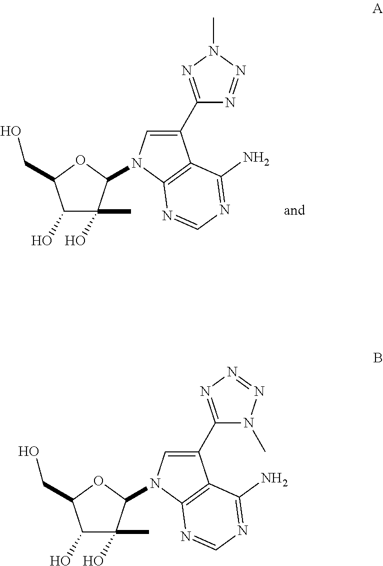

### Step A: 7-(2-C-methyl-β-D-ribofuranosyl)-5-(1H-tetrazol-5-yl)-7H-pyrrolo[2,3-d]pyrimidin-4-amine

Azidotributyltin (6 eq) was added to a 0.15 M solution of 4-amino-7-(2-C-methyl-β-D-ribofuranosyl)-7H-pyrrolo[2,3-d]pyrimidine-5-carbonitrile (Ding Y. et al., Bioorganic and Medicinal Chemistry Letters 2005, 15, 725) in a 6:1 v:v mixture of toluene and DMF, and the resulting mixture was heated for 30 minutes at 130° C. under microwave irradiation. The solution was allowed to cool to RT, diluted with a 1.25 M solution of HCl in MeOH and concentrated under reduced pressure. The residue was purified by preparative RP-HPLC eluting with MeCN/water containing 0.1% TFA to give the title compound as a solid (77%). ¹H NMR (300 MHz, DMSO-d₆) δ 8.38 (s, 1H), 8.28 (s, 1H), 6.27 (s, 1H), 3.98 (m, 1H), 3.93-3.84 (m, 2H), 3.78 (m, 1H), 0.80 (s, 3H); MS (ES⁺) C₁₃H₁₆N₈O₄ requires: 348, found: 349 [M+H]⁺.

### Step B: 7-(2-C-methyl-β-D-ribofuranosyl)-5-(1-methyl-1H-tetrazol-5-yl)-7H-pyrrolo[2,3-d]pyrimidin-4-amine (A) and 7-(2-C-methyl-β-D-ribofuranosyl)-5-(2-methyl-2H-tetrazol-5-yl)-7H-pyrrolo[2,3-d]pyrimidin-4-amine (B)

Iodomethane (2.0 eq) was added to a 0.1 M solution of 7-(2-C-methyl-β-D-ribofuranosyl)-5-(1H-tetrazol-5-yl)-7H-pyrrolo[2,3-d]pyrimidin-4-amine from step A and K₂CO₃ (1.05 eq) in 1:1 v:v mixture of acetone and DMF. The resulting mixture was stirred at RT for 3 h, diluted with a 1.25 M solution of HCl in MeOH and the volatiles were removed under reduced pressure. The residue was purified by preparative RP-HPLC eluting with MeCN/water containing 0.1% TFA to give a the title products A and B as a solid in a 2:1 mixture (22%). ¹H NMR (300 MHz, DMSO-d₆) δ 8.77 (s, 1HB), 8.65 (s, 1HA) 8.38 (s, 1HA), 8.36 (s, 1H B), 6.27 (s, 1HB), 6.25 (s, 1HA), 4.46 (s, 3HA), 4.25 (s, 3HB), 4.14 (d, J=9.0 Hz, 1HB), 4.04 (d, J=9.0 Hz, 1HA), 4.01-3.83 (m, 2HA+B), 3.80-3.65 (m, 1HA+B), 0.80 (s, 3HB), 0.76 (s, 3HA); MS (ES⁺) C₁₄H₁₈N₈O₄ requires: 362, found: 363 [M+H]⁺.

### Example 2

### Entry 2, Table 1

### 7-(2-C-methyl-β-D-ribofuranosyl)-5-(1,3-oxazol-2-yl)-7H-pyrrolo[2,3-d]pyrimidin-4-amine

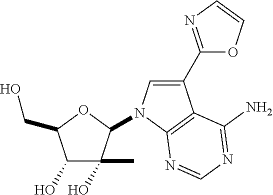

PdCl₂(PPh₃)₂ (0.1 eq) was added to a 0.1 M solution of 5-iodo-7-(2-C-methyl-β-D-ribofuranosyl)-7H-pyrrolo[2,3-d]pyrimidin-4-amine and 2-(tri-n-butylstannyl)oxazole (3.0 eq) in DMF and the resulting mixture was heated at 120° C. for 3 h under microwave irradiation. The reaction mixture was cooled to RT, partitioned between water/hexanes and the water phase was purified by preparative RP-HPLC eluting with MeCN/water containing 0.1% TFA to give the title compound as a solid (11%). ¹H NMR (300 MHz, DMSO-d₆) δ 9.88 (bs, 1H), 8.60 (s, 1H), 8.36 (s, 1H), 8.33 (bs, 1H), 8.23 (s, 1H), 7.45 (s, 1H), 6.21 (s, 1H), 4.06 (d, J=9.6 Hz, 1H), 3.99-3.87 (m, 2H), 3.73 (m, 1H), 0.77 (s, 3H); MS (ES⁺) C₁₅H₁₇N₅O₅ requires: 347, found: 348 [M+H]⁺.

### Example 3

### Entry 3, Table 1

### 7-(2-C-methyl-β-D-ribofuranosyl)-5-pyrimidin-2-yl-7H-pyrrolo[2,3-d]pyrimidin-4-amine

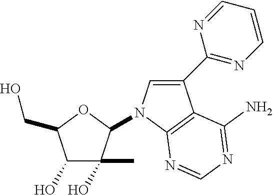

The title compound was obtained in 17% isolated yield following the same procedure described for example 3, using 2-tributylstannylpyrimidine instead of 2-(tri-n-butylstannyl)oxazole. Microwave irradiation time was reduced to 40 min. ¹H NMR (300 MHz, DMSO-d₆, 300 K) δ 10.79 (bs, 1H), 8.89 (d, J=5.0 Hz, 2H), 8.82 (s, 1H), 8.48 (bs, 1H), 8.40 (s, 1H), 7.43 (t, J=5.0 Hz, 1H), 6.26 (s, 1H), 4.04 (d, J=9.1 Hz, 1H), 3.99-3.86 (m, 2H), 3.71 (m, 1H), 0.78 (s, 3H); MS (ES⁺) C₁₆H₁₈N₆O₄ requires: 358, found: 359 [M+H]⁺.

### Example 4

### Entry 7, Table 1

### 7-(2-C-methyl-β-D-ribofuranosyl)-5-(1,2,4-oxadiazol-3-yl)-7H-pyrrolo[2,3-d]pyrimidin-4-amine

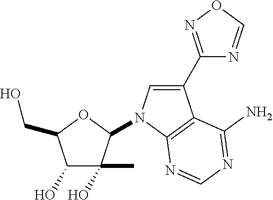

Hydroxylamine hydrochloride (1.5 eq) and triethylamine (2.0 eq) were added to a 0.2 M solution of 4-amino-7-(2-C-methyl-β-D-ribofuranosyl)-7H-pyrrolo[2,3-d]pyrimidine-5-carbonitrile (Ding Y. et al., Bioorganic and Medicinal Chemistry Letters 2005, 15, 725) in ethanol. The resulting mixture was heated at 50° C. for 6 h, cooled to RT and concentrated under reduced pressure to yield 4-amino-N′-hydroxy-7-(2-C-methyl-β-D-ribofuranosyl)-7H-pyrrolo[2,3-d]pyrimidine-5-carboximidamide as a solid [MS (ES⁺) C₁₃H₁₈N₆O₅ requires: 338.1, found: 339 [M+H]⁺]. The solid residue was suspended in triethyl orthoformate (3.0 eq), treated with BF₃.Et₂O (0.3 eq) and heated at 100° C. for 15 min. After cooling to RT, the mixture was diluted with water and concentrated under reduced pressure. The residue was diluted with DCM (0.1 M), cooled to 0° C. and treated with a 1 M solution of BBr₃ in DCM (6.0 eq). After stirring for 3 h at RT the reaction mixture was diluted at 0° C. with MeOH, treated with a 2M solution of ammonia in MeOH and concentrated under reduced pressure. The residue was purified by preparative RP-HPLC eluting with MeCN/water containing 0.1% TFA to give the title compound as a solid (25%). ¹H NMR (300 MHz, DMSO-d₆) δ 9.78 (s, 1H), 8.76 (s, 1H), 8.62-8.04 (m, 2H), 8.37 (s, 1H), 6.23 (s, 1H), 4.04 (d, J=9.2 Hz, 1H), 3.99-3.85 (m, 2H), 3.72 (m, 1H), 0.77 (s, 3H); MS (ES⁺) C₁₄H₁₆N₆O₅ requires: 348, found: 349 [M+H]⁺.

### Example 5

### Entry 5, Table 1

### 7-(2-C-methyl-β-D-ribofuranosyl)-5-(1H-pyrazol-5-yl)-7H-pyrrolo[2,3-d]pyrimidin-4-amine

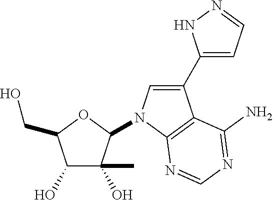

Pd(PPh₃)₄ (0.1 eq) was added to a 0.1 M solution of 5-iodo-7-(2-C-methyl-β-D-ribofuranosyl)-7H-pyrrolo[2,3-d]pyrimidin-4-amine, 1H-pyrazole-5-boronic acid (1.5 eq) and Na₂CO₃ (2M aq. solution, 15 eq) in dioxane. The reaction mixture was heated at 120° C. for 500 seconds under microwave irradiation and then filtered through a pad of celite. The filtrate was concentrated under reduced pressure and the residue was purified by preparative RP-HPLC eluting with MeCN/water containing 0.1% TFA to give the title compound as a solid (65%). ¹H NMR (300 MHz, DMSO-d₆) δ 13.14 (s, 1H), 10.65 (bs, 1H), 8.60 (bs, 1H), 8.40 (s, 1H), 8.38 (s, 1H), 7.93 (bs, 1H), 6.63 (bs, 1H), 6.20 (s, 1H), 4.04 (d, J=9.1 Hz, 1H), 3.99-3.87 (m, 2H), 3.75 (m, 1H), 0.78 (s, 3H); MS (ES⁺) C₁₅H₁₈N₆O₄ requires: 346, found: 347 [M+H]⁺.

### Example 6

### Entry 4, Table 1

### 5-(2-methoxy-1,3-thiazol-4-yl)-7-(2-C-methyl-β-D-ribofuranosyl)-7H-pyrrolo[2,3-d]pyrimidin-4-amine trifluoroacetate

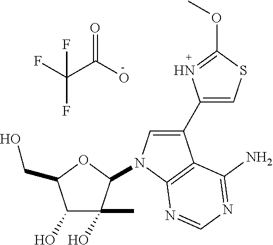

The title compound was obtained in 16% isolated yield following the same procedure described for example 5, using 2-methoxy-4-(tributylstannyl)thiazole instead of 1H-pyrazole-5-boronic acid. ¹H NMR (300 MHz, DMSO-d₆) δ 8.38 (s, 1H), 8.36 (s, 1H), 7.29 (s, 1H), 6.22 (s, 1H), 4.14 (s, 3H), 4.02 (d, J=9.8 Hz, 1H), 3.98-3.86 (m, 2H), 3.80-3.70 (m, 1H), 0.78 (s, 3H); ¹⁹F NMR (300 MHz, DMSO-d₆) δ −73.88; MS (ES⁺) C₁₆H₁₉N₅O₅S requires: 393, found: 394 [M+H]⁺.

### Example 7

### Entry 6, Table 1

### 7-(2-C-methyl-β-D-ribofuranosyl)-5-(1H-pyrazol-4-yl)-7H-pyrrolo[2,3-d]pyrimidin-4-amine

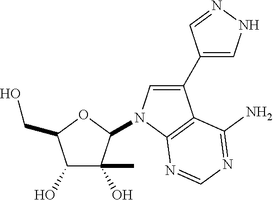

The title compound was obtained in 55% isolated yield following the same procedure described for example 5, using 1H-pyrazole-2-boronic acid instead of 1H-pyrazole-5-boronic acid. Microwave irradiation time was prolonged to 1200 sec. ¹H NMR (300 MHz, DMSO-d₆) δ 8.44 (s, 1H), 7.91, (s, 1H), 7.73 (m, 1H), 7.80 (s, 1H), 6.22 (s, 1H), 4.02 (d, J=9.3 Hz, 1H), 3.96-3.81 (m, 2H), 3.69 (m, 1H), 0.78 (s, 3H); MS (ES⁺) C₁₅H₁₈N₆O₄ requires: 346, found: 347 [M+H]⁺.

### Example 8

### Entry 8, Table 1

### 7-(2-C-methyl-β-D-ribofuranosyl)-5-(1-methyl-1H-1,2,3-triazol-4-yl)-7H-pyrrolo[2,3-d]pyrimidin-4-amine trifluoroacetate

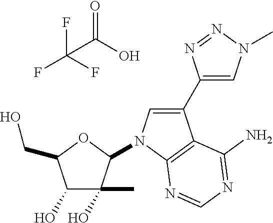

Trimethylsilylmethyl azide (3.0 eq) was added to a 0.25 M solution of 5-ethynyl-7-(2-C-methyl-β-D-ribofuranosyl)-7H-pyrrolo[2,3-d]-pyrimidin-4-amine in a 1:1 v:v mixture of water and ^{t}BuOH containing L-ascorbic acid sodium salt (0.5 eq) and copper(II) sulfate pentahydrate (0.05 eq). The heterogeneous mixture was stirred at 50° C. overnight, cooled to RT and concentrated under reduced pressure. The residue was treated with 1M aq. solution of NaOH (5.0 eq) in a 1:1 v:v mixture of MeOH and H₂O. The resulting mixture was stirred at 50° C. for 2 h and then concentrated under reduced pressure. Purification by preparative RP-HPLC eluting with MeCN/water containing 0.1% TFA gave the title compound as a solid (21%). ¹H NMR (300 MHz, DMSO-d₆) δ 8.48 (s, 1H), 8.40 (s, 1H), 8.20 (s, 1H), 6.23 (s, 1H), 4.17 (s, 3H), 3.98-3.85 (m, 3H), 3.75 (m, 1H), 0.79 (s, 3H); ¹⁹F NMR (300 MHz, DMSO-d₆) δ −73.90; MS (ES⁺) C₁₅H₁₉N₇O₄ requires: 361, found: 362 [M+H]⁺.

### Example 9

### Entry 21, Table 1

### 7-(2-deoxy-2-fluoro-2-methyl-β-D-ribofuranosyl)-5-(1H-pyrazol-5-yl)-7H-pyrrolo[2,3-d]pyrimidin-4-amine

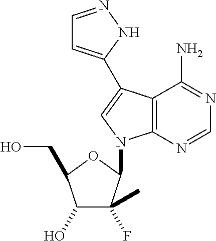

### Step A: 7-(2-deoxy-2-fluoro-2-methyl-β-D-ribofuranosyl)-5-iodo-7H-pyrrolo[2,3-d]pyrimidin-4-amine

DIAD (2.8 eq) was added to a 0.05 M solution of Ph₃P (3.0 eq) in acetonitrile and the resulting mixture was stirred for 30 minutes at 0° C. The solution was then added via cannula into a 0.1 M solution of 4-chloro-5-iodo-7H-pyrrolo[2,3-d]pyrimidine (2.2 eq) and 3,5-di-O-benzoyl-2-deoxy-2-fluoro-2-methyl-D-ribofuranose (1.0 eq) in acetonitrile at −40° C. The resulting pale brown suspension was stirred overnight at RT. The reaction was quenched with AcOEt and the organic phase was washed with water, brine and dried over Na₂SO₄. The volatiles were removed under reduced pressure and the residue was purified by flash chromatography (gradient elution from 0% to 5% AcOEt/Hexane then isocratic elution 5% AcOEt/Hexane) to obtain 4-chloro-7-(3,5-di-O-benzoyl-2-deoxy-2-fluoro-2-methyl-β-D-ribofuranosyl)-5-iodo-7H-pyrrolo[2,3-d]pyrimidine as the first eluting anomer. The latter was dissolved in a 7M solution of NH₃ in MeOH (0.01 M) and the resulting mixture was stirred overnight at 110° C. in a closed vessel. The volatiles were then removed under reduced pressure and the residue was purified by flash chromatography eluting with MeOH:DCM 1:9 to give the title compound as white foam (24%). ¹H NMR (400 MHz, CD₃CN/D₂O) δ 8.14 (s, 1H), 7.61 (s, 1H), 6.35 (d, J=18.5 Hz, 1H), 4.20 (dd, J=24.0, 9.4 Hz, 1H), 3.98-3.95 (m, 2H), 3.78 (dd, J=12.0, 2.8 Hz, 1H), 1.00 (d, J=22.6 Hz, 3H); ¹⁹F-NMR (400 MHz, CD₃CN/D₂O) δ −160.89; MS (ES⁺) C₁₂H₄FIN₄O₃ requires: 408, found: 409 (M+H⁺).

### Step B: 7-(2-deoxy-2-fluoro-2-methyl-β-D-ribofuranosyl)-5-(1H-pyrazol-5-yl)-7H-pyrrolo[2,3-d]pyrimidin-4-amine

The title compound was obtained following the same procedure described for example 5, using 7-(2-deoxy-2-fluoro-2-methyl-β-D-ribofuranosyl)-5-iodo-7H-pyrrolo[2,3-d]pyrimidin-4-amine instead of 5-iodo-7-(2-C-methyl-β-D-ribofuranosyl)-7H-pyrrolo[2,3-d]pyrimidin-4-amine (68%). ¹H NMR (400 MHz, CD₃CN/D₂O) δ 8.19 (bd, J=17.1 Hz, 2H), 7.72 (bs, 1H), 6.63 (bs, 1H), 6.47 (d, J=17.1 Hz, 1H), 4.26 (bd, J=25.1 Hz, 1H), 4.05 (bs, 2H), 3.87 (m, 1H), 1.08 (d, J=22.4 Hz, 3H); ¹⁹F-NMR (400 MHz, CD₃CN/D₂O) δ −162.64; MS (ES⁺) C₁₅H₁₇FN₅O₃ requires: 348, found: 349 (M+H⁺).

### Example 10

### Entry 22, Table 1

### 7-(2-deoxy-2-fluoro-2-methyl-β-D-ribofuranosyl)-5-(1,3-oxazol-2-yl)-7H-pyrrolo[2,3-d]pyrimidin-4-amine

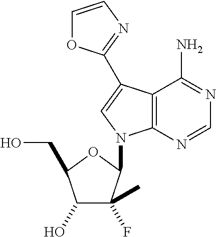

The title compound obtained in 20% isolated yield following the same procedure described for example 2, using 7-(2-deoxy-2-fluoro-2-methyl-β-D-ribofuranosyl)-5-iodo-7H-pyrrolo[2,3-d]pyrimidin-4-amine instead of 5-iodo-7-(2-C-methyl-β-D-ribofuranosyl)-7H-pyrrolo[2,3-d]pyrimidin-4-amine. ¹H NMR (300 MHz, CD₃CN/D₂O) δ 8.43 (s, 1H), 8.26 (s, 1H), 7.78 (s, 1H), 7.27 (s, 1H), 6.45 (d, J=17.1 Hz, 1H), 4.23 (dd, J=24.0, 9.5 Hz, 1H), 4.06-3.99 (m, 2H), 3.83 (dd, J=12.0, 2.4 Hz, 1H), 1.06 (d, J=22.8 Hz, 3H); ¹⁹F-NMR (300 MHz, CD₃CN/D₂O) δ −163.14; MS (ES⁺) C₁₅H₁₆FN₅O₄ requires: 349, found: 350 (M+H⁺).

### Example 11

### Entry 23, Table 1

### 7-(2-deoxy-2-fluoro-2-methyl-β-D-ribofuranosyl)-5-(1,2,4-oxadiazol-3-yl)-7H-pyrrolo[2,3-d]pyrimidin-4-amine

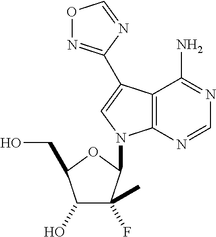

The title compound was obtained in 27% isolated yield following the same procedure described for example 4, using 4-amino-7-(2-deoxy-2-fluoro-2-methyl-β-D-ribofuranosyl)-7H-pyrrolo[2,3-d]pyrimidine-5-carbonitrile instead of 4-amino-7-(2-C-methyl-β-D-ribofuranosyl)-7H-pyrrolo[2,3-d]pyrimidine-5-carbonitrile. ¹H-NMR (400 MHz, CD₃CN/D₂O) δ 9.07 (s, 1H), 8.53 (s, 1H), 8.18 (s, 1H), 6.38 (d, J=16.9 Hz, 1H), 4.13 (dd, J=24.0, 8.2 Hz, 1H), 3.94-3.88 (m, 2H), 3.71 (m, 1H), 0.96 (d, J=22.6 Hz, 1H); ¹⁹F-NMR (400 MHz, CD₃CN/D₂O) δ −163.26; MS (ES⁺) C₁₄H₁₅FN₆O₄ requires: 350, found: 351 (M+H⁺).

### Example 12

### Entry 10, Table 1

### 7-(2-C-methyl-5-triphospho-β-D-ribofuranosyl)-5-(1-methyl-1H-tetrazol-5-yl)-7H-pyrrolo[2,3-d]pyrimidin-4-amine and 7-(2-C-methyl-5-triphospho-β-D-ribofuranosyl)-5-(2-methyl-2H-tetrazol-5-yl)-7H-pyrrolo[2,3-d]pyrimidin-4-amine

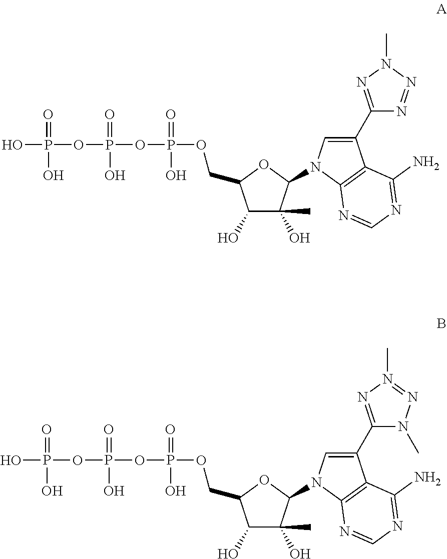

A:B=2:1

¹H NMR (300 MHz, D₂O, 300 K) δ 8.61-8.16 (m, 1HA+1HB), 8.02 (s, 1HA), 8.00 (s, 1HB), 6.33 (s, 1HB), 6.25 (s, 1HA), 4.60 (m, 1HA or 1HB), 4.55-4.39 (m, 1HA+1HB), 4.38-4.27 (m, 1HA+1HB), 4.17 (m, 1HA or 1HB), 3.28-3.08 (m, 6H), 3.05-2.76 (m, 18H), 1.85-1.64 (m, 6H), 1.50-1.25 (m, 18H), 1.00-0.80 (m, 12H); ³¹P NMR (121 MHz, D₂O, 300 K) δ −10.08-−11.26 (m, 2PA+2PB), −22.70-−23.56 (m, 1PA+1PB); MS (ES⁻) C₁₄H₂₁N₈O₁₃P₃ requires: 602.0, found: 601 [M−H]⁻, 623 [M+Na—H]⁻.

### Example 13

### Entry 11, Table 1

### 7-(2-C-methyl-5-triphospho-β-D-ribofuranosyl)-5-(1,3-oxazol-2-yl)-7H-pyrrolo[2,3-d]pyrimidin-4-amine

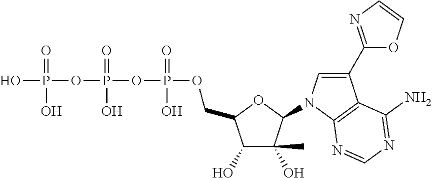

¹H NMR (300 MHz, D₂O, 300 K) δ 8.37 (m, 1H), 8.03-7.91 (m, 2H), 7.18 (s, 1H), 6.16 (s, 1H), 4.67 (m, 1H), 4.44 (m, 1H), 4.32 (m, 1H), 4.19 (m, 1H), 3.26-3.10 (m, 6H), 3.02-2.81 (m, 18H), 1.86-1.66 (m, 6H), 1.50-1.26 (m, 18H), 1.00-0.86 (m, 9H), 0.81 (s, 3H); ³¹P NMR (121 MHz, D₂O, 300 K) δ −10.49 (d, J=19.4 Hz, 1P), −11.07 (d, J=19.4 Hz, 1P), −23.08 (t, J=19.4 Hz, 1P); MS (ES⁻) C₁₅H₂₀N₅O₁₄P₃ requires: 587.0, found: 586 [M−H]⁻, 608 [M+Na—H]⁻.

### Example 14

### Entry 12, Table 1

### 7-(2-C-methyl-5-triphospho-β-D-ribofuranosyl)-5-pyrimidin-2-yl-7H-pyrrolo[2,3-d]pyrimidin-4-amine

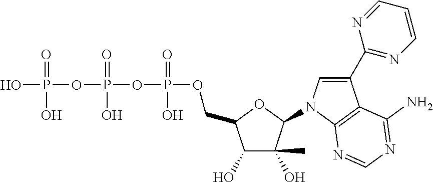

¹H NMR (300 MHz, D₂O, 300 K) δ 8.66 (bs, 2H), 8.35 (bs, 1H), 8.06 (s, 1H), 7.34 (bs, 1H), 6.12 (s, 1H), 4.61 (m, 1H), 4.47 (m, 1H), 4.32 (m, 1H), 4.05 (d, J=9.4 Hz, 1H), 3.21-3.09 (m, 6H), 2.99-2.81 (m, 18H), 1.80-1.66 (m, 6H), 1.46-1.25 (m, 18H), 0.96-0.83 (m, 9H), 0.74 (s, 3H); ³¹P NMR (121 MHz, D₂O, 300 K) δ −10.46 (d, J=18.6 Hz, 1P), −10.75 (d, J=19.6 Hz, 1P), −22.84 (bt, J=19.1 Hz, 1P); MS (ES⁻) C₁₆H₂₁N₆O₁₃P₃ requires: 598.0, found: 597 [M−H]⁻, 619 [M+Na—H]⁻

### Example 15

### Entry 13, Table 1

### 5-(2-methoxy-1,3-thiazol-4-yl)-7-(2-C-methyl-5-triphospho-β-D-ribofuranosyl)-7H-pyrrolo[2,3-d]pyrimidin-4-amine

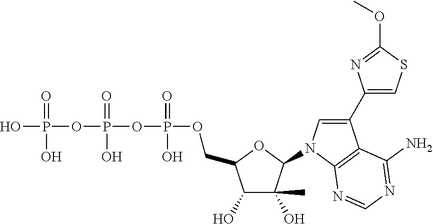

¹H NMR (300 MHz, D₂O, 300 K) δ 8.30 (s, 1H), 7.85 (s, 1H), 7.66 (s, 1H), 6.08 (s, 1H), 4.78 (m, 1H), 4.42 (m, 1H), 4.29 (d, J=9.1 Hz, 1H), 4.17 (d, J=9.1 Hz, 1H), 4.06 (s, 3H), 3.25-3.09 (m, 6H), 2.92 (s, 18H), 1.83-1.67 (m, 6H), 1.48-1.26 (m, 18H), 0.98-0.85 (m, 9H), 0.72 (s, 3H); ³¹P NMR (121 MHz, D₂O, 300 K) δ −10.50 (d, J=19.7 Hz, 1P), −11.26 (d, J=19.7 Hz, 1P), −22.97 (t, J=19.7 Hz, 1P); MS (ES⁻) C₁₆H₂₂N₅O₁₄P₃S requires: 633.0, found: 632 [M−H]⁻, 654 [M+Na—H]⁻

### Example 16

### Entry 14, Table 1

### 7-(2-C-methyl-5-triphospho-β-D-ribofuranosyl)-5-(1H-pyrazol-5-yl)-7H-pyrrolo[2,3-d]pyrimidin-4-amine

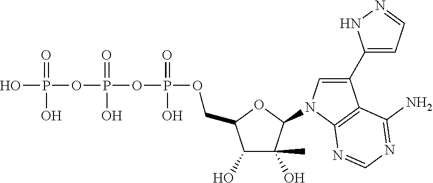

¹H NMR (300 MHz, D₂O, 300 K) δ 8.25 (bs, 1H), 7.79 (s, 1H), 7.71 (s, 1H), 7.00 (s, 1H), 6.11 (s, 1H), 4.71 (m, 1H), 4.43 (m, 1H), 4.25 (m, 2H), 3.16 (m, 6H), 2.91 (m, 18H), 1.83-1.66 (m, 6H), 1.47-1.27 (m, 18H), 0.97-0.85 (m, 9H), 0.77 (s, 3H); ³¹P NMR (121 MHz, D₂O, 300 K) δ −10.23 (d, J=19.5 Hz, 1P), −11.13 (d, J=19.5 Hz, 1P), −22.90 (t, J=19.5 Hz, 1P); MS (ES⁻) C₁₅H₂₁N₆O₁₃P₃ requires: 586.0, found: 585 [M−H]⁻, 607 [M+Na—H]⁻.

### Example 17

### Entry 15, Table 1

### 7-(2-C-methyl-5-triphospho-β-D-ribofuranosyl)-5-(1H-pyrazol-4-yl)-7H-pyrrolo[2,3-d]pyrimidin-4-amine

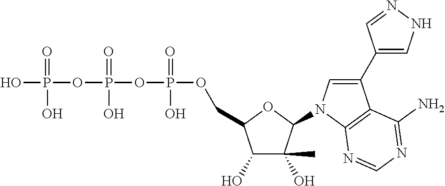

¹H NMR (300 MHz, D₂O, 300 K) δ 8.34 (m, 1H), 7.82 (bs, 2H), 7.51 (s, 1H), 6.23 (s, 1H), 4.56 (m, 1H), 4.45-4.13 (m, 3H), 3.14 (m, 6H), 2.89 (m, 18H), 1.83-1.61 (m, 6H), 1.48-1.19 (m, 18H), 0.98-0.86 (m, 9H), 0.84 (s, 3H); ³¹P NMR (121 MHz, D₂O, 300 K) δ −10.50 (d, J=19.3 Hz, 1P), −11.20 (d, J=19.3 Hz, 1P), −23.02 (t, J=19.3 Hz, 1P); MS (ES⁻) C₁₅H₂₁N₆O₁₃P₃ requires: 586.0, found: 585 [M−H]⁻, 607 [M+Na—H]⁻.

### Example 18

### Entry 16, Table 1

### 7-(2-C-methyl-5-triphospho-β-D-ribofuranosyl)-5-(1,2,4-oxadiazol-3-yl)-7H-pyrrolo[2,3-d]pyrimidin-4-amine

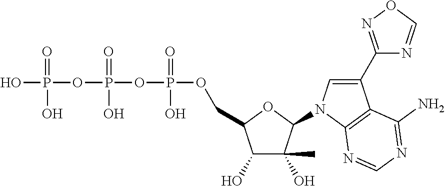

10% yield; 50% pure

¹H NMR (300 MHz, D₂O, 300 K) δ 9.33 (s, 1H), 8.41 (m, 1H), 8.09 (s, 1H), 6.23 (s, 1H), 4.61 (m, 1H), 4.55 (m, 1H), 4.33 (m, 1H), 4.16 (d, J=9.4 Hz, 1H), 3.17 (m, 6H), 2.92 (m, 18H), 1.82-1.68 (m, 6H), 1.48-1.29 (m, 18H), 0.98-0.87 (m, 9H), 0.84 (s, 3H); ³¹P NMR (121 MHz, D₂O, 300 K) δ −10.45-−11.15 (m, 2P), −22.92 (t, J=19.6 Hz, 1P); MS (ES⁻) C₁₄H₉N₆O₁₄P₃ requires: 588.0, found: 587 [M−H]⁻

### Example 19

### Entry 17, Table 1

### 7-(2-C-methyl-5-triphospho-β-D-ribofuranosyl)-5-(1-methyl-1H-1,2,3-triazol-4-yl)-7H-pyrrolo[2,3-d]pyrimidin-4-amine

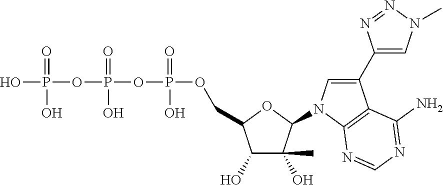

¹H NMR (300 MHz, D₂O, 300 K) δ 8.63 (s, 1H), 8.29 (m, 1H), 7.88 (s, 1H), 6.10 (s, 1H), 4.71 (m, 1H), 4.42 (m, 1H), 4.31-4.14 (m, 2H), 4.20 (s, 3H), 3.26-3.06 (m, 6H), 2.91 (m, 18H), 1.82-1.64 (m, 6H), 1.47-1.25 (m, 18H), 0.96-0.84 (m, 9H), 0.75 (s, 3H); ³¹P NMR (121 MHz, D₂O, 300 K) δ −9.46 (d, J=19.9 Hz, 1P), −11.18 (d, J=19.9 Hz, 1P), −22.70 (t, J=19.9 Hz, 1P); MS (ES⁻) C₁₅H₂₂N₇O₁₃P₃ requires: 601.0, found: 600 [M−H]⁻.

### Example 20

### Entry 18, Table 1

### 7-(2-deoxy-2-fluoro-2-methyl-5-triphospho-β-D-ribofuranosyl)-5-(1,2,4-oxadiazol-3-yl)-7H-pyrrolo[2,3-d]pyrimidin-4-amine

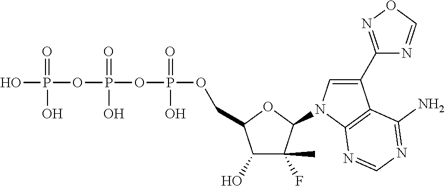

¹H NMR (300 MHz, D₂O, 300 K) δ 9.29 (s, 1H), 8.34 (bs, 1H), 8.03 (s, 1H), 6.42 (d, J=18.6 Hz, 1H), 4.61 (m, 1H), 4.44 (m, 1H), 4.31 (m, 1H), 3.14 (m, 6H), 2.89 (m, 18H), 1.78-1.66 (m, 6H), 1.43-1.27 (m, 18H), 1.04 (d, J=23.1 Hz, 3H), 0.95-0.84 (m, 9H); ³¹P NMR (121 MHz, D₂O, 300 K) δ −10.60 (d, J=19.4 Hz, 1P), −11.12 (d, J=19.4 Hz, 1P), −22.99 (t, J=19.4 Hz, 1H); ¹⁹F NMR (282 MHz, D₂O, 300 K) δ −161.35 (s, 1F); MS (ES⁻) C₁₄H₁₈N₆O₁₃P₃ requires: 590.0, found: 589 [M−H]⁻, 611 [M+Na—H]⁻

### Example 21

### Entry 19, Table 1

### 7-(2-deoxy-2-fluoro-2-methyl-5-triphospho-β-D-ribofuranosyl)-5-(1,3-oxazol-2-yl)-7H-pyrrolo[2,3-d]pyrimidin-4-amine

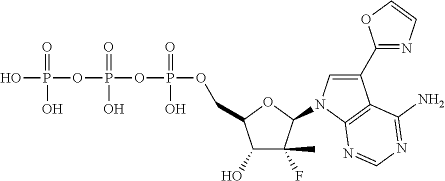

¹H NMR (300 MHz, D₂O, 300 K) δ 8.28 (bs, 1H), 7.99 (s, 1H), 7.92 (s, 1H), 7.21 (s, 1H), 6.43 (d, J=17.8 Hz, 1H), 4.63 (m, 1H), 4.52-4.29 (m, 3H), 3.22-3.07 (m, 6H), 2.90 (m, 18H), 1.80-1.64 (m, 6H), 1.44-1.26 (m, 18H), 1.06 (d, J=22.8 Hz, 3H), 0.96-0.83 (m, 9H); ³¹P NMR (121 MHz, D₂O, 300 K) δ −10.36 (d, J=19.6 Hz, 1P), −11.23 (d, J=19.6 Hz, 1P), −23.07 (t, J=19.6 Hz, 1P); ¹⁹F NMR (282 MHz, D₂O, 300 K) δ −162.22 (s, 1F); MS (ES⁻) C₁₅H₁₉FN₅O₁₃P₃ requires: 589.0, found: 588 [M−H]⁻

### Example 22

### Entry 20, Table 1

### 7-(2-deoxy-2-fluoro-2-methyl-5-triphospho-β-D-ribofuranosyl)-5-(1H-pyrazol-5-yl)-7H-pyrrolo[2,3-d]pyrimidin-4-amine

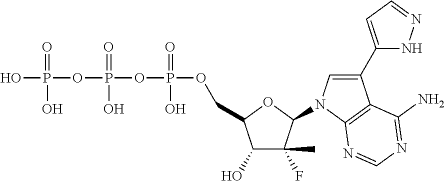

¹H NMR (300 MHz, D₂O, 300 K) δ 8.23 (bs, 1H), 7.74 (s, 1H), 7.70 (s, 1H), 6.96 (s, 1H), 6.30 (d, J=17.2 Hz, 1H), 4.70 (m, 1H), 4.50-4.25 (m, 3H), 3.14 (m, 6H), 2.89 (m, 18H), 1.80-1.63 (m, 6H), 1.45-1.26 (m, 18H), 0.98 (d, J=22.6 Hz, 3H), 0.94-0.82 (m, 9H); ³¹P NMR (121 MHz, D₂O, 300 K) δ −9.88 (d, J=19.4 Hz, 1P), −11.24 (d, J=19.4 Hz, 1P), −22.82 (t, J=19.4 Hz, 1P); ¹⁹F NMR (282 MHz, D₂O, 300 K) δ −162.93 (s, 1F); MS (ES⁻) C₁₅H₂₀FN₆O₁₂P₃ requires: 588.0, found: 587 [M−H]⁻;

### Example 23

### Entry 25, Table 1

### 1-(2-C-methyl-β-D-ribofuranosyl)-3-(1H-pyrazol-3-yl)-1H-pyrazolo[3,4-d]pyrimidin-4-amine

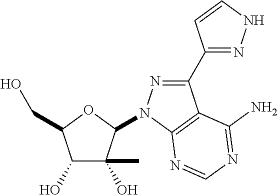

1-(2-C-methyl-β-D-ribofuranosyl)-3-iodo-1H-pyrazolo[3,4-d]pyrimidin-4-amine (prepared according to the procedure described in J. Med. Chem. 1993, 36 (22), 3424-3430) was dissolved in a 1:1:1 v/v/v mixture of dioxane/CH₃CN/H₂O to give a 0.07 M solution. 1H-Pyrazol-5-ylboronic acid (2.0 eq), K₂CO₃ (2.6 eq), and Pd(PPh₃)₄ (0.06 eq) were added under argon and the resulting reaction mixture was stirred at reflux for 10 hrs. The mixture was then cooled to RT, diluted with water and extracted several times with DCM. The combined organic extracts were dried (Na₂SO₄) and concentrated under reduced pressure. The residue was dissolved in MeOH to give a 0.07 M solution and 2M aq. NaOH (1.2 eq) was added. After stirring 30 min at RT, the reaction mixture was neutralized by addition of 2M aq. HCl until pH 7 was reached. The title compound was obtained in 28% yield over 2 steps after HPLC purification; ¹H NMR (400 MHz, DMSO-d₆): 13.34 (1H, br s), 9.28 (1H, br s), 8.22 (1H, s), 8.05 (1H, br s), 7.96 (1H, d, J=2.4 Hz), 6.75 (1H, d, J=2.4 Hz), 6.18 (1H, s), 5.17 (1H, s), 5.08 (1H, d, J=7.3 Hz), 4.76-4.79 (1H, m), 4.26 (1H, t, J=8.2 Hz), 3.97-4.01 (1H, m), 3.68-3.81 (2H, m), 0.80 (3H, s). MS (ES⁺) C₁₄H₁₇N₇O₄ requires: 347, found: 348 (M+H⁺).

### Example 24

### Entry 26, Table 1

### 1-[5-O-(hydroxy{[hydroxy(phosphonooxy)phosphoryl]oxy}phosphoryl)-2-C-methyl-b-D-ribofuranosyl]-3-(1H-pyrazol-3-yl)-1H-pyrazolo[3,4-d]pyrimidin-4-amine

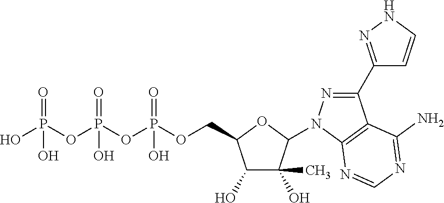

¹H NMR (300 MHz, D2O, 300 K) δ 8.23 (s, 1H), 7.83 (bs, 1H), 7.01 (bs, 1H), 6.32 (s, 1H), 4.64-4.41 (m, 4H), 0.98 (s, 3H); ³¹P NMR (121 MHz, D₂O, 300 K) δ −10.55 (d, J=20.4 Hz, 1P), −10.95 (d, J=20.4 Hz, 1P), −23.00 (t, J=20.4 Hz, 1P); MS (ES−) C14H20N7O13P3 requires: 587.3, found: 586 [M−H].

### Example 25

### Entry 27, Table 1

### 7-(2-C-methyl-β-D-ribofuranosyl)-5-(1,3,4-oxadiazol-2-yl)-7H-pyrrolo[2,3-d]pyrimidin-4-amine

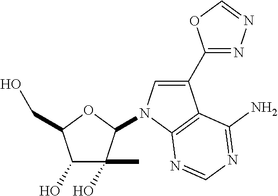

### Step 1: 4-amino-7-(2,3,5-tri-O-acetyl-2-C-methyl-β-D-ribofuranosyl)-7H-pyrrolo[2,3-d]pyrimidine-5-carboxylic acid

A 2.0 M solution of sodium chlorite (10 eq) and sodium phosphate monobasic (7.5 eq) in water was added dropwise at 0° C. to a 0.05 M solution of 4-amino-7-(2,3,5-tri-O-acetyl-2-C-methyl-β-D-ribofuranosyl)-7H-pyrrolo[2,3-d]pyrimidine-5-carbaldehyde (prepared according to the procedure reported by Watanabe, S. et al., Nucleosides and Nucleotides, 1983, 2, 113 and Seela, F. et al., Synthesis, 1997, 1067) in t-butanol containing 2-methyl-2-butene (2 M in THF, 20 eq). The resulting reaction mixture was stirred at RT overnight. The volatiles were removed under reduced pressure and the residue was diluted with water and extracted with DCM. The combined organic layers were washed with brine, dried over Na2SO4 and concentrated to dryness under reduced pressure. The residue was purified by Silica gel chromatography eluting with 0% to 5% MeOH in DCM to afford the title compound as a white solid (90%); ¹H NMR (300 MHz, DMSO-d₆) δ 8.19 (s, 1H), 8.00 (s, 1H), 6.61 (s, 1H), 5.53 (d, J=6.4 Hz, 1H), 4.48-4.32 (m, 3H), 2.16-2.06 (m, 9H), 1.33 (s, 3H); MS (ES⁺) C₁₉H₂₂N₄O₉ requires 450.4, found 451 [M+H]⁺.

### Step 2: 4-amino-7-(2-C-methyl-β-D-ribofuranosyl)-7H-pyrrolo[2,3-d]pyrimidine-5-carbohydrazide

4-methylmorpholine (1.0 eq) was added to a cooled 0° C., stirred 0.2 M solution of 4-amino-7-(2,3,5-tri-O-acetyl-2-C-methyl-β-D-ribofuranosyl)-7H-pyrrolo[2,3-d]pyrimidine-5-carboxylic acid in THF containing tert-butyl hydrazinecarboxylate (1.0 eq) and 1H-benzotriazol-1-ol hydrate (2.0 eq). After 5 minutes stirring at 0° C., N,N-dicyclohexylcarbodiimide (1.0 eq) was added. The reaction mixture was stirred 1 h at 0° C. and 12 h at RT; it was then cooled to 0° C. and filtered through a short pad of Celite. The filtrate was diluted with DCM, washed with aqueous s.s. of NaHCO₃, brine and dried over Na₂SO₄. The residue was evaporated to dryness under reduced pressure to yield 4-amino-N′-(tert-butoxycarbonyl)-7-(2,3,5-tri-O-acetyl-2-C-methyl-β-D-ribofuranosyl)-7H-pyrrolo[2,3-d]pyrimidine-5-carbohydrazide an off white solid; MS (ES+) C₂₄H₃₂N₆O₁₀ requires: 564.65, found: 565 [M+H]⁺.

The solid residue was dissolved in 4M HCl in dioxane (25 eq) and stirred at RT for 6 h. The reaction mixture was evaporated under reduced pressure and partitioned between water/Et₂O. The pH was neutralised with aqueous s.s. NaHCO₃ and extracted. The water layers were evaporated in vacuo to give a residue that was purified by RP-HPLC (Atlantis T3, 19×150 mm, 5 um) eluting with MeCN/water containing 0.1% TFA to give the title compound as a white solid (67%); ¹H NMR (300 MHz, DMSO-d₆) δ 11.00-10.63 (bs, 1H), 9.06-8.49 (bs, 1H), 8.38 (s, 1H), 8.22 (s, 1H), 6.24 (s, 1H), 4.01-3.81 (m, 3H), 3.74 (dd, J=5.1, 12.6 Hz, 1H), 0.79 (s, 3H); MS (ES⁺) C₁₃H₁₈N₆O₅ requires 338.32, found 339 [M+H]⁺

### Step 3: 7-(2-C-methyl-β-D-ribofuranosyl)-5-(1,3,4-oxadiazol-2-yl)-7H-pyrrolo[2,3-d]pyrimidin-4-amine

BF₃.Et₂O (0.2 eq) was dropwise added to a stirred 0.5 M solution of 4-amino-7-(2-C-methyl-(3-D-ribofuranosyl)-7H-pyrrolo[2,3-d]pyrimidine-5-carbohydrazide (1.0 eq) in DMF containing triethyl orthoformate (2.0 eq). The reaction mixture was heated at 70° C. for 180 min to yield a 1:1 mixture of the target molecule and the 7-[2,3-O-(ethoxymethylidene)-2-C-methyl-β-D-ribofuranosyl]-5-(1,3,4-oxadiazol-2-yl)-7H-pyrrolo[2,3-d]pyrimidin-4-amine intermediate. The reaction mixture was then cooled to RT, diluted with water and treated with aqueous 1M HCl up to pH ˜2 for 20 minutes and then it was treated with aqueous 6M NH₄OH (up to pH˜8) and stirred for 30 minutes at RT. The reaction mixture was then evaporated under reduced pressure and purified by preparative RP-HPLC eluting with MeCN/water containing 0.1% TFA to give the title compound (25%) as a white fluffy solid. ¹H NMR (300 MHz, DMSO-d₆) δ 9.34 (s, 1H), 8.66 (s, 1H), 8.31 (s, 1H), 6.22 (s, 1H), 4.17-3.82 (m, 3H), 3.78-3.67 (m, 1H), 0.78 (s, 3H); (MS (ES⁺) C₁₄H₁₆N₆O₅ requires 348.31, found 349 [M+H]⁺

### Example 26

### Entry 28, Table 1

### 7-(2-C-methyl-5-triphospho-β-D-ribofuranosyl)-5-(1,3,4-oxadiazol-2-yl)-7H-pyrrolo[2,3-d]pyrimidin-4-amine

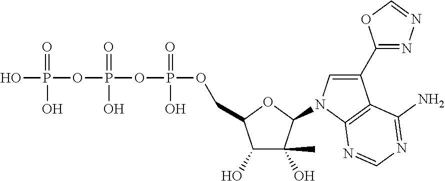

¹H NMR (300 MHz, D₂O, 300 K) δ 9.02 (s, 1H), 8.42-8.32 (bs, 1H), 8.22 (s, 1H), 6.28 (s, 1H), 4.69-4.57 (m, 1H), 4.53-4.40 (m, 1H), 4.39-4.23 (m, 2H), 3.24-3.12 (m, 6H), 2.94 (s, 12H), 1.85-1.67 (m, 6H), 1.49-1.28 (m, 18H), 0.99-0.84 (m, 12H); ³¹P NMR (121 MHz, D₂O, 300 K) δ −10.34 (d, J=18.0 Hz, 1P), −11.14 (d, J=20.6 Hz, 1P), −22.91 (t, J=18.6 Hz, 1P); MS (ES⁻) C₁₄H₁₉N₆O₁₄P₃ requires: 588.0, found: 587 [M−H]⁻

### Example 27

### Entry 29, Table 1

### Ethyl 2-{[(R)-({(2R,3S,4R,5R)-5-[4-amino-5-(1H-pyrazol-5-yl)-7H-pyrrolo[2,3-d]pyrimidin-7-yl]-3,4-dihydroxy-4-methyltetrahydrofuran-2-yl}methoxy)(phenoxy)phosphoryl]amino}propanoate

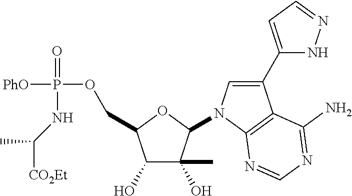

To a 0.1 M solution of 7-(2-C-methyl-β-D-ribofuranosyl)-5-(1H-pyrazol-5-yl)-7H-pyrrolo[2,3-d]pyrimidin-4-amine (Example 5) in dry THF at −78° C. was added dropwise t-BuMgCl (1.0 M solution in THF, 2.2 eq.). The reaction mixture was stirred at −78° C. for 5 min and then at 0° C. for further 30 min. L-alanine-N-chlorophenoxyphosphinyl-, ethyl ester (McGuigan, C. et al., J. Med. Chem., 2005, 48, 3504) was then added dropwise (1.0 M solution in dry THF, 1.5 eq.) at 0° C., and the resulting mixture was stirred at RT for 60 min and quenched with 2 ml of ss NH₄Cl. The volatiles were removed under reduced pressure and the residue was purified by RP-HPLC (Atlantis T3, 19×150 mm, 5 um) eluting with MeCN/H₂O containing 0.1% TFA to give the title compound. ¹H NMR (600 MHz, CD₃CN+D₂O, 300 K) δ 11.86 (bs, 1H), 10.87 (bs, 1H), 8.96 (s, 1H), 8.18 (s, 1H), 7.86-7.82 (m, 1H), 7.58-7.54 (m, 1H), 7.36-7.31 (m, 2H), 7.25-7.15 (m, 3H), 6.81-6.76 (m, 1H), 6.32-6.30 (m, 1H), 4.55-4.49 (m, 1H), 4.47-4.40 (m, 1H), 4.18-4.13 (m, 1H), 4.18-3.97 (m, 4H), 3.96-3.90 (m, 1H), 1.27-1.25 (m, 3H), 1.14-1.12 (m, 3H), 0.85 (s, 3H). ³¹P (243 MHz. CD₃CN+D₂O) δ 3.85, 3.49. MS (ES⁺) C₂₆H₃₂N₇O₈P requires 601.2, found 602 [M+H]⁺

### Example 28

### Entry 30, Table 1

### Ethyl 2-{[(R)-({(2R,3S,4R,5R)-5-[4-amino-5-(1,3-oxazol-2-yl)-7H-pyrrolo[2,3-d]pyrimidin-7-yl]-3,4-dihydroxy-4-methyltetrahydrofuran-2-yl}methoxy)(phenoxy)phosphoryl]amino}propanoate

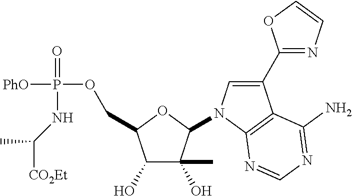

The title compound was prepared as described for Example 27, employing 7-(2-C-methyl-β-D-ribofuranosyl)-5-(1,3-oxazol-2-yl)-7H-pyrrolo[2,3-d]pyrimidin-4-amine (Example 2) instead of 7-(2-C-methyl-β-D-ribofuranosyl)-5-(1H-pyrazol-5-yl)-7H-pyrrolo[2,3-d]pyrimidin-4-amine (Example 5). ¹H NMR (300 MHz, CD₃CN+D₂O, 300 K) δ 8.24 (bs, 1H), 8.03-8.01 (m, 1H), 7.60 (s, 0.5H), 7.51 (s, 0.5H), 7.36-7.33 (m, 2H), 7.23-7.17 (m, 6H), 6.29-6.28 (m, 1H), 4.56-4.34 (m, 2H), 4.19-4.06 (m, 5H), 1.33-1.30 (m, 2H), 1.26-1.24 (m, 2H), 1.17-1.11 (m, 4H), 0.85 (s, 1.5H), 0.83 (s, 1.5H). ³¹P (300 MHz. CD₃CN+D₂O, 300K) δ 3.72, 3.64. MS (ES⁺) C₂₇H₃₁N₆O₉P requires 602.2, found 603 [M+H]⁺

The following Table 1 lists specific compounds of the present invention. The table provides the structure and name of each compound and the observed mass as determined via ES-MS, either as its molecular ion plus H (M+1) or as its molecular ion minus H (M−1) for positive and negative ionization mode respectively. Molecular ion plus Na plus H (M+22+1) and molecular ion plus Na minus H (M+22−1) are also reported when observed. The synthetic scheme employed to prepare the compound is indicated in the last column.

TABLE 1

| | | Exact | Observed | |
|---|---|---|---|---|
| # | Name | Mass | Mass | Scheme |
| 1 | 7-(2-C-methyl-β-D-ribofuranosyl)-5-(1-methyl-1H- | 362.1 | 363 | 3 |
| | tetrazol-5-yl)-7H-pyrrolo [2,3-d]pyrimidin-4-amine | | | |
| | and 7-(2-C-methyl-β-D-ribofuranosyl)-5-(2-methyl- | | | |
| | 2H-tetrazol-5-yl)-7H-pyrrolo[2,3-d]pyrimidin-4- | | | |
| | amine | | | |
| 2 | 7-(2-C-methyl-β-D-ribofuranosyl)-5-(1,3-oxazol-2- | 347.1 | 348 | 1 |
| | yl)-7H-pyrrolo[2,3-d]pyrimidin-4-amine | | | |
| 3 | 7-(2-C-methyl-β-D-ribofuranosyl)-5-pyrimidin-2-yl- | 358.1 | 359, 381 | 1 |
| | 7H-pyrrolo[2,3-d]pyrimidin-4-amine | | [M + Na + H]⁺ | |
| 4 | 5-(2-methoxy-1,3-thiazol-4-yl)-7-(2-C-methyl-β-D- | 393.1 | 394 | 1 |
| | ribofuranosyl)-7H-pyrrolo[2,3-d]pyrimidin-4-amine | | | |
| | trifluoroacetate | | | |
| 5 | 7-(2-C-methyl-β-D-ribofuranosyl)-5-(1H-pyrazol-5- | 346.1 | 347 | 1 |
| | yl)-7H-pyrrolo[2,3-d]pyrimidin-4-amine | | | |
| 6 | 7-(2-C-methyl-β-D-ribofuranosyl)-5-(1H-pyrazol-4- | 346.1 | 347 | 1 |
| | yl)-7H-pyrrolo[2,3-d]pyrimidin-4-amine | | | |
| 7 | 7-(2-C-methyl-β-D-ribofuranosyl)-5-(1,2,4- | 348.1 | 349 | 4 |
| | oxadiazol-3-yl)-7H-pyrrolo[2,3-d]pyrimidin-4-amine | | | |
| 8 | 7-(2-C-methyl-β-D-ribofuranosyl)-5-(1-methyl-1H- | 361.2 | 362 | 3 |
| | 1,2,3-triazol-4-yl)-7H-pyrrolo[2,3-d]pyrimidin-4- | | | |
| | amine trifluoroacetate | | | |
| 9 | 7-(2-C-methyl-β-D-riboruranosyl)-5-(2-thienyl)-7H- | 362.1 | 363 | 1 |
| | pyrrolo [2,3-d]pyrimidin-4-amine | | | |
| 10 | 7-(2-C-methyl-5-triphospho-β-D-ribofuranosyl)-5-(1- | 602.0 | 601, 623 | 5 |
| | methyl-1H-tetrazol-5-yl)-7H-pyrrolo[2,3-d]pyrimidin- | | [M + Na − H]⁻ | |
| | 4-amine and 7-(2-C-methyl-5-triphospho-β-D- | | | |
| | ribofuranosyl)-5-(2-methyl-2H-tetrazol-5-yl)-7H- | | | |
| | pyrrolo [2,3-d]pyrimidin-4-amine | | | |
| 11 | 7-(2-C-methyl-5-triphospho-β-D-ribofuranosyl)-5- | 587.0 | 586, 608 | 5 |
| | (1,3-oxazol-2-yl)-7H-pyrrolo[2,3-d]pyrimidin-4- | | [M + Na − H]⁻ | |
| | amine | | | |
| 12 | 7-(2-C-methyl-5-triphospho-β-D-ribofuranosyl)-5- | 598.0 | 597, 619 | 5 |
| | pyrimidin-2-yl-7H-pyrrolo[2,3-d]pyrimidin-4-amine | | [M + Na − H]⁻ | |
| 13 | 5-(2-methoxy-1,3-thiazol-4-yl)-7-(2-C-methyl-5- | 633.0 | 632, 654 | 5 |
| | triphospho-β-D-ribofuranosyl)-7H-pyrrolo[2,3- | | [M + Na − H]⁻ | |
| | d]pyrimidin-4-amine | | | |
| 14 | 7-(2-C-methyl-5-triphospho-β-D-ribofuranosyl)-5- | 586.0 | 585, 607 | 5 |
| | (1H-pyrazol-5-yl)-7H-pyrrolo[2,3-d]pyrimidin-4- | | [M + Na − H]⁻ | |
| | amine | | | |
| 15 | 7-(2-C-methyl-5-triphospho-β-D-ribofuranosyl)-5- | 586.0 | 585, 607 | 5 |
| | (1H-pyrazol-4-yl)-7H-pyrrolo[2,3-d]pyrimidin-4- | | [M + Na − H]⁻ | |
| | amine | | | |
| 16 | 7-(2-C-methyl-5-triphospho-β-D-ribofuranosyl)-5- | 588.0 | 587 | 5 |
| | (1,2,4-oxadiazol-3-yl)-7H-pyrrolo[2,3-d]pyrimidin-4- | | | |
| | amine | | | |
| 17 | 7-(2-C-methyl-5-triphospho-β-D-ribofuranosyl)-5-(1- | 601.0 | 600 | 5 |
| | methyl-1H-1,2,3-triazol-4-yl)-7H-pyrrolo[2,3- | | | |
| | d]pyrimidin-4-amine | | | |
| 18 | 7-(2-deoxy-2-fluoro-2-methyl-5-triphospho-β-D- | 590.0 | 589, 611 | 5 |
| | ribofuranosyl)-5-(1,2,4-oxadiazol-3-yl)-7H- | | [M + Na − H]⁻ | |
| | pyrrolo[2,3-d]pyrimidin-4-amine | | | |
| 19 | 7-(2-deoxy-2-fluoro-2-methyl-5-triphospho-β-D- | 589.0 | 588 | 5 |
| | ribofuranosyl)-5-(1,3-oxazol-2-yl)-7H-pyrrolo[2,3- | | | |
| | d]pyrimidin-4-amine | | | |
| 20 | 7-(2-deoxy-2-fluoro-2-methyl-5-triphospho-β-D- | 588.0 | 587 | 5 |
| | ribofuranosyl)-5-(1H-pyrazol-5-yl)-7H-pyrrolo[2,3- | | | |
| | d]pyrimidin-4-amine | | | |
| 21 | 7-(2-deoxy-2-fluoro-2-methyl-β-D-ribofuranosyl)-5- | 348.1 | 349 | 1 |
| | (1H-pyrazol-5-yl)-7H-pyrrolo[2,3-d]pyrimidin-4- | | | |
| | amine | | | |
| 22 | 7-(2-deoxy-2-fluoro-2-methyl-β-D-ribofuranosyl)-5- | 349.1 | 350 | 1 |
| | (1,3-oxazol-2-yl)-7H-pyrrolo[2,3-d]pyrimidin-4- | | | |
| | amine | | | |
| 23 | 7-(2-deoxy-2-fluoro-2-methyl-β-D-ribofuranosyl)-5- | 350.1 | 351 | 4 |
| | (1,2,4-oxadiazol-3-yl)-7H-pyrrolo[2,3-d]pyrimidin-4- | | | |
| | amine | | | |
| 24 | 7-(2-C-methyl-β-D-ribofuranosyl)-5-(6- | 387.1 | 388 | 1 |
| | methoxypyridin-3-yl)-7H-pyrrolo[2,3-d]pyrimidin-4- | | | |
| | amine | | | |
| 25 | 1-(2-C-methyl-β-D-ribofuranosyl)-3-(1H-pyrazol-3- | 347.1 | 348 | 1 |
| | yl)-1H-pyrazolo[3,4-d]pyrimidin-4-amine | | | |
| 26 | 1-[5-O- | 587.3 | 586 | 5 |
| | (hydroxy{[hydroxy(phosphonooxy)phosphoryl]oxy}phos- | | | |
| | phoryl)-2-C-methyl-β-D-ribofuranosyl]-3-(1H- | | | |
| | pyrazol-3-yl)-1H-pyrazolo[3,4-d]pyrimidin-4-amine | | | |
| 27 | 7-(2-C-methyl-β-D-ribofuranosyl)-5-(1,3,4- | 348.3 | 349 | 6 |
| | oxadiazol-2-yl)-7H-pyrrolo[2,3-d]pyrimidin-4-amine | | | |
| 28 | 7-(2-C-methyl-5-triphospho-β-D-ribofuranosyl)-5- | 588.0 | 587 | 5 |
| | (1,3,4-oxadiazol-2-yl)-7H-pyrrolo[2,3-d]pyrimidin-4- | | | |
| | amine | | | |
| 29 | Ethyl 2-{[(R)-({(2R,3S,4R,5R)-5-[4-amino-5-(1H- | 601.2 | 602 | 7 |
| | pyrazol-5-yl)-7H-pyrrolo[2,3-d]pyrimidin-7-yl]-3,4- | | | |
| | dihydroxy-4-methyltetrahydrofuran-2- | | | |
| | yl}methoxy)(phenoxy)phosphoryl]amino}propanoate | | | |
| 30 | Ethyl 2-{[(R)-({(2R, 3S, 4R,5R)-5-[4-amino-5-(1,3- | 602.2 | 603 | 7 |
| | oxazol-2-yl)-7H-pyrrolo[2,3-d]pyrimidin-7-yl]-3,4- | | | |
| | dihydroxy-4-methyltetrahydrofuran-2- | | | |
| | yl}methoxy)(phenoxy)phosphoryl]amino}propanoate | | | |

### Biological Assays

The assays employed to measure the inhibition of HCV NS5B polymerase and HCV replication are described below.

The effectiveness of the compounds of the present invention as inhibitors of HCV NS5B RNA-dependent RNA polymerase (RdRp) was measured in the following assay.

### A. Assay for Inhibition of HCV NS5B Polymerase:

This assay was used to measure the ability of the nucleoside derivatives of the present invention to inhibit the enzymatic activity of the RNA-dependent RNA polymerase (NS5B) of the hepatitis C virus (HCV) on a heteromeric RNA template.

### Procedure:

### Assay Buffer Conditions: (52.5 μL-total/reaction)

20 mM Tris, pH 7.5

45 mM KCl

2 mM MgCl2

0.01% Triton X-100

1 μg BSA, DNase Free

1 mM DTT

2 nM DC55-lb.BK or 10 nM DC55-2b.2

20 nM heterogeneous template dCoh

UTP 1 uM

ATP 1 uM

CTP 1 uM

GTP 1 uM

3H-UTP 1,000,000 cpm

2.5 μl/reaction inhibitor compound in H₂O

The compounds were tested at various concentrations up to 100 μM final concentration. Nucleoside derivatives were pipetted into wells of a 96-well plate. The enzyme diluted in the reaction buffer was pipetted into the wells and incubated at room temperature for 10 minutes; then the template dCoh was added and incubated for 10 minutes at room temperature. The reaction was initiated by addition of a mixture of nucleotide triphosphates (NTP's), including the radiolabeled UTP, and allowed to proceed at room temperature for 2 hours. Blank samples were done omitting the dCoh template. The reaction was quenched by addition of 50 ul TCA 20% (trichloroacetic acid)/NaPPi 20 mM and the plates were put in ice for 5 minutes. Then, the mixtures were filtered onto Unifilter GF/B 96-well plates (PerkinElmer), washed with TCA 2.5%. 50 ul/well of scintillator solution (Microscint 20, PerkinElmer) were added and the plates were counted in a scintillator counter.

The percentage of inhibition was calculated according to the following equation:

% Inhibition=[1−(cpm in test reaction−cpm in blank)/(cpm in control reaction−cpm in blank)]×100.

Representative compounds were tested in the HCV NS5B polymerase assay and results are reported as IC50 activity ranges in Table 2

TABLE 2

| | | IC₅₀ |
|---|---|---|
| Compound | Structure | (μM) |
| 10 | 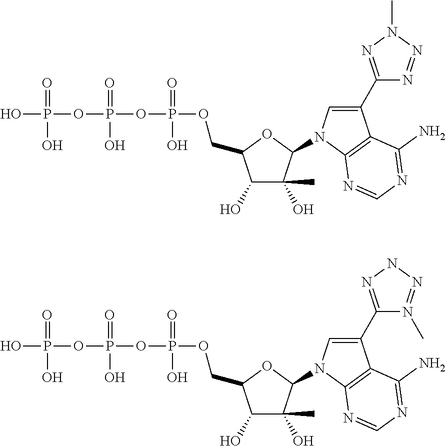 | ++ |
| 11 | 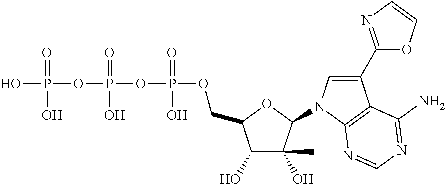 | +++ |
| 12 | 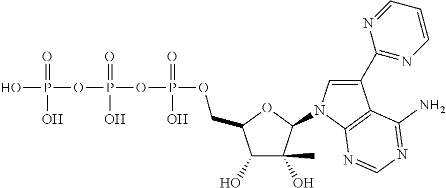 | ++ |
| 13 | 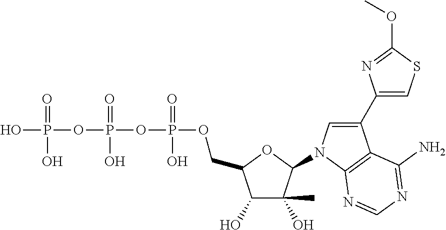 | ++ |
| 14 | 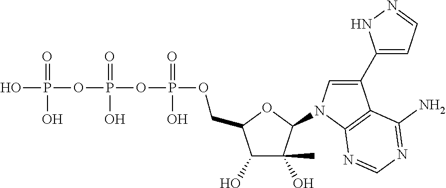 | +++ |
| 15 | 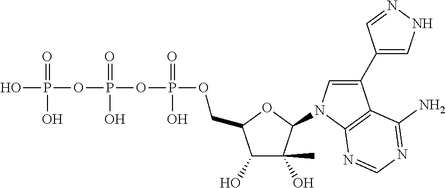 | ++ |
| 16 | 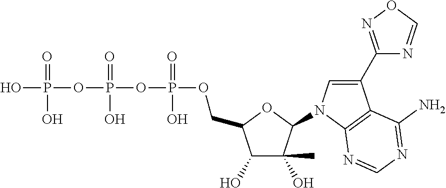 | +++ |
| 17 | 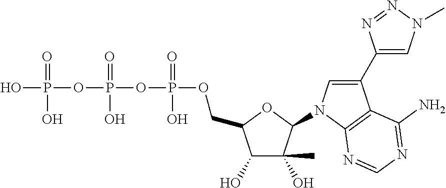 | ++ |
| 18 | 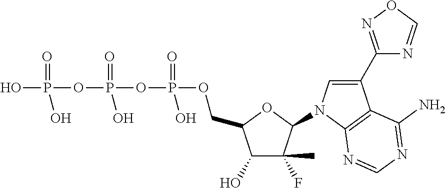 | +++ |
| 19 | 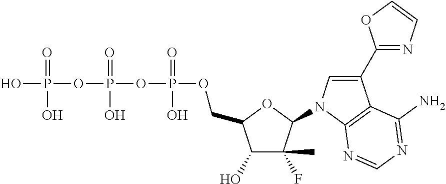 | +++ |
| 20 | 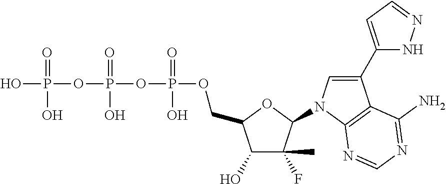 | +++ |
| 26 | 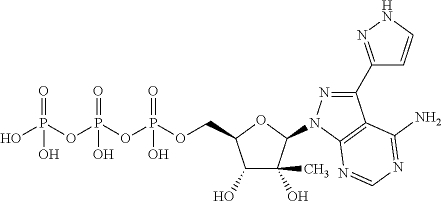 | ++ |
| 28 | 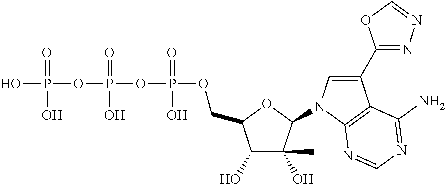 | ++ |

Activity ranges:
+++: <1 μM; ++: <50 μM; +: >50 μM;

### B. Assay for Inhibition of HCV RNA Replication:

The compounds of the present invention were also evaluated for their ability to affect the replication of Hepatitis C Virus RNA in cultured hepatoma (HuH-7) cells containing a subgenomic HCV Replicon. The details of the assay are described below. This Replicon assay is a modification of that described in V. Lohmann, F. Korner, J-O. Koch, U. Herian, L. Theilmann, and R. Bartenschlager, “Replication of a Sub-genomic Hepatitis C Virus RNAs in a Hepatoma Cell Line,” Science 285:110 (1999).

### Protocol:

The assay was an in situ Ribonuclease protection, Scintillation Proximity based-plate assay (SPA). 10,000-40,000 cells were plated in 100-200 μL of media containing 0.8 mg/mL G418 in 96-well cytostar plates (Amersham). Compounds were added to cells at various concentrations up to 100 μM in 1% DMSO at time 0 to 18 h and then cultured for 24-96 h. Cells were fixed (20 min, 10% formalin), permeabilized (20 min, 0.25% Triton X-100/PBS) and hybridized (overnight, 50° C.) with a single-stranded ³³P RNA probe complementary to the (+) strand NS5B (or other genes) contained in the RNA viral genome. Cells were washed, treated with RNAse, washed, heated to 65° C. and counted in a Top-Count Inhibition of replication was read as a decrease in counts per minute (cpm).

Human HuH-7 hepatoma cells, which were selected to contain a subgenomic replicon, carry a cytoplasmic RNA consisting of an HCV 5′ non-translated region (NTR), a neomycin selectable marker, an EMCV IRES (internal ribosome entry site), and HCV non-structural proteins NS3 through NS5B, followed by the 3′ NTR.

Representative compounds were tested in the HCV replication assay and results are reported as IC50 activity ranges in Table 3

TABLE 3

| Com- | | EC₅₀ |
|---|---|---|
| pound | Structure | (μM) |
| 1 | 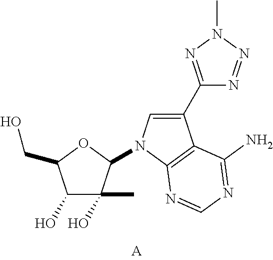 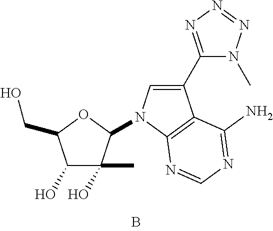 | +++ |
| 2 | 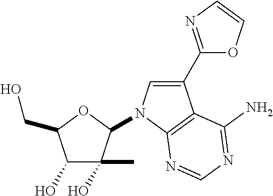 | +++ |
| 3 | 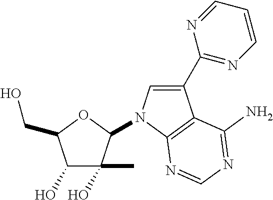 | + |
| 4 | 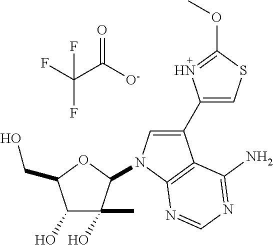 | + |
| 5 | 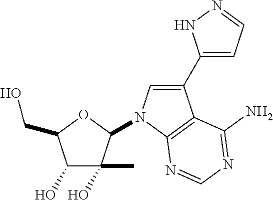 | ++ |
| 6 | 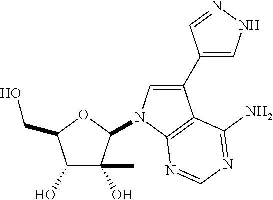 | ++ |
| 7 | 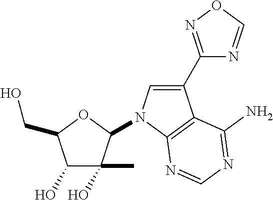 | +++ |
| 8 | 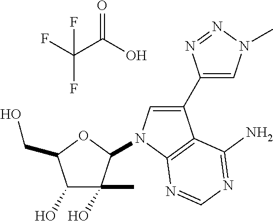 | ++ |
| 9 | 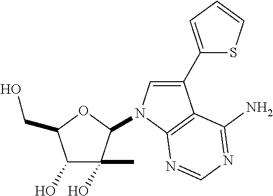 | +++ |
| 21 | 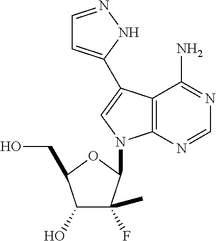 | ++ |
| 22 | 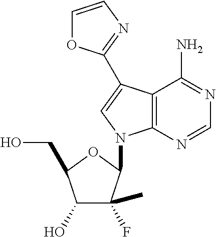 | ++ |
| 23 | 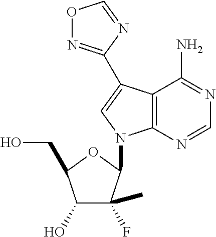 | ++ |
| 24 | 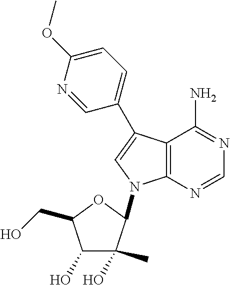 | + |
| 25 | 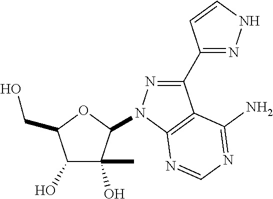 | +++ |
| 27 | 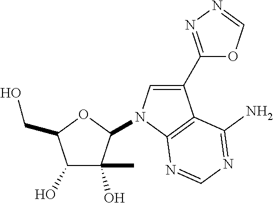 | +++ |
| 29 | 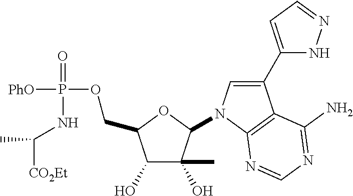 | +++ |
| 30 | 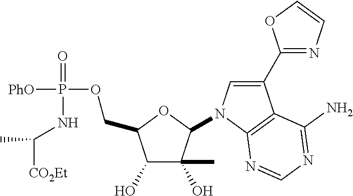 | +++ |

Activity ranges:
+++: <20 μM; ++: 20-50 μM; +: >50 μM;

### Example of a Pharmaceutical Formulation

As a specific embodiment of an oral composition of a compound of the present invention, 50 mg of any one of the Examples is formulated with sufficient finely divided lactose to provide a total amount of 580 to 590 mg to fill a size O hard gelatin capsule.

While the invention has been described and illustrated in reference to specific embodiments thereof, those skilled in the art will appreciate that various changes, modifications, and substitutions can be made therein without departing from the spirit and scope of the invention. For example, effective dosages other than the preferred doses as set forth hereinabove may be applicable as a consequence of variations in the responsiveness of the human being treated for severity of the HCV infection. Likewise, the pharmacologic response observed may vary according to and depending upon the particular active compound selected or whether there are present pharmaceutical carriers, as well as the type of formulation and mode of administration employed, and such expected variations or differences in the results are contemplated in accordance with the objects and practices of the present invention. It is intended therefore that the invention be limited only by the scope of the claims which follow and that such claims be interpreted as broadly as is reasonable.

1. Compounds of structural formula (I): 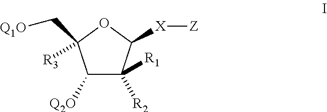
and pharmaceutically acceptable salts thereof; wherein:
X is an optionally substituted basic ring system found in nucleosides and nucleotide analogues X being linked to the carbohydrate ring through a N atom of the basic ring system;
Z is a 5 or 6 membered heterocyclic ring, containing one to three heteroatoms optionally substituted by an oxo, S(O)ₙ, S(O)ₙR⁴, C₁₋₄ alkyl, C₁₋₄ haloalkyl, CH₂OR⁴, CO₂R⁴, CONR⁴R⁵, NR⁴C(O)R⁵ or NR⁴R⁵ groups wherein R⁴ and R⁵ are independently selected from hydrogen and C₁₋₄ alkyl; and Z is attached to a ring atom of X that is two ring atoms from the N atom that links X to the carbohydrate ring;
R¹ is hydrogen, hydroxy, halo or C₁₋₆alkyl optionally substituted by fluoro;
R² is hydroxy, halo, OMe, C₁-C₁₆-alkylcarbonyl or hydrogen;
R³ is hydrogen or an azido, ethynyl, cyano or a C₁₋₆ aliphatic group optionally substituted by fluoro;
Q¹ is hydrogen or a mono-, di- or tri-phosphate group or a protecting group Q³ and
Q² is hydrogen or a protecting group Q⁴.

2. A compound according to claim 1 wherein X is a purine, pyrrolopyrimidine, pyrazolopyrimidine or pyrimidine ring optionally substituted by halo, one or more oxo or hydroxy groups, or by one or more amino groups optionally substituted by COR⁶, wherein R⁶ is a C₁₋₆ aliphatic group or phenyl.

3. A compound according to claim 2 wherein Z is a 5 membered heterocyclic ring that contains two or three heteroatoms selected from oxygen and nitrogen, of which at most one is oxygen.

4. A compound according to claim 3 wherein R¹ is methyl or fluorine; R² is hydroxy or fluoro; and R³ is hydrogen.

5. A compound according to claim 4 wherein Q¹ is selected from hydrogen, monophosphate, diphosphate, or triphosphate, or C₁-C₁₆-alkylcarbonyl or a monophosphate prodrug residue 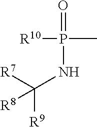
wherein R⁷ is hydrogen, methyl or benzyl; more suitably hydrogen or methyl; R⁸ is hydrogen or methyl; more suitably hydrogen; R⁹ is Ph, CO₂R¹¹ or CR¹³R¹⁴OC(O)R¹² and R¹⁰ is hydroxyl or OR¹⁶; wherein R¹⁶ is an aromatic or heteroaromatic ring or CH₂CH₂SR¹⁷, where R¹⁷ is C₁-C₆ alkylcarbonyl, optionally substituted with a hydroxyl group.

6. A compound according to claim 5 wherein Q¹ is hydrogen or triphosphoryl.

7. A compound according to claim 6 wherein Q² is selected from hydrogen, C₁-C₁₆-alkylcarbonyl or an amino acyl residue of the structure: 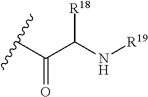
wherein R¹⁸ is hydrogen or C₁-C₅ alkyl and R¹⁹ is hydrogen.

8. A compound according to claim 1 wherein the compound is selected from the formula (II), (III), (IV) and (V): 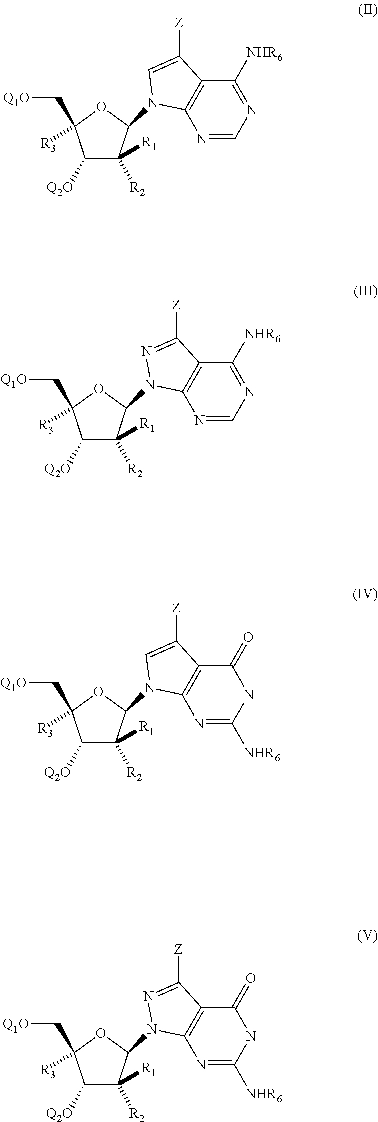
and pharmaceutically acceptable salts thereof; wherein R¹ to R⁶, Z, Q¹ and Q² are as hereinbefore defined.

9. A compound according to claim 1 wherein the compound is selected from:
5-(2-methoxy-1,3-thiazol-4-yl)-7-(2-C-methyl-β-D-ribofuranosyl)-7H-pyrrolo[2,3-d]pyrimidin-4-amine,
7-(2-C-methyl-β-D-ribofuranosyl)-5-pyrimidin-2-yl-7H-pyrrolo[2,3-d]pyrimidin-4-amine,
7-(2-C-methyl-β-D-ribofuranosyl)-5-(1,3-oxazol-2-yl)-7H-pyrrolo[2,3-d]pyrimidin-4-amine,
7-(2-C-methyl-β-D-ribofuranosyl)-5-(1H-pyrazol-5-yl)-7H-pyrrolo[2,3-d]pyrimidin-4-amine,
7-(2-C-methyl-β-D-ribofuranosyl)-5-(1H-pyrazol-4-yl)-7H-pyrrolo[2,3-d]pyrimidin-4-amine,
7-(2-C-methyl-β-D-ribofuranosyl)-5-(1,2,4-oxadiazol-3-yl)-7H-pyrrolo[2,3-d]pyrimidin-4-amine,
7-(2-C-methyl-β-D-ribofuranosyl)-5-(1-methyl-1H-1,2,3-triazol-4-yl)-7H-pyrrolo[2,3-d]pyrimidin-4-amine,
7-(2-C-methyl-β-D-ribofuranosyl)-5-(2-thienyl)-7H-pyrrolo[2,3-d]pyrimidin-4-amine,
7-(2-C-methyl-5-triphospho-β-D-ribofuranosyl)-5-(1-methyl-1H-tetrazol-5-yl)-7H-pyrrolo[2,3-d]pyrimidin-4-amine,
7-(2-C-methyl-5-triphospho-β-D-ribofuranosyl)-5-(2-methyl-2H-tetrazol-5-yl)-7H-pyrrolo[2,3-d]pyrimidin-4-amine,
7-(2-C-methyl-5-triphospho-β-D-ribofuranosyl)-5-(1,3-oxazol-2-yl)-7H-pyrrolo[2,3-d]pyrimidin-4-amine,
5-(2-methoxy-1,3-thiazol-4-yl)-7-(2-C-methyl-5-triphospho-β-D-ribofuranosyl)-7H-pyrrolo[2,3-d]pyrimidin-4-amine,
7-(2-C-methyl-5-triphospho-β-D-ribofuranosyl)-5-(1H-pyrazol-5-yl)-7H-pyrrolo[2,3-d]pyrimidin-4-amine,
7-(2-C-methyl-5-triphospho-β-D-ribofuranosyl)-5-(1H-pyrazol-4-yl)-7H-pyrrolo[2,3-d]pyrimidin-4-amine,
7-(2-C-methyl-5-triphospho-β-D-ribofuranosyl)-5-(1,2,4-oxadiazol-3-yl)-7H-pyrrolo[2,3-d]pyrimidin-4-amine,
7-(2-C-methyl-5-triphospho-β-D-ribofuranosyl)-5-(1-methyl-1H-1,2,3-triazol-4-yl)-7H-pyrrolo[2,3-d]pyrimidin-4-amine,
7-(2-deoxy-2-fluoro-2-methyl-5-triphospho-β-D-ribofuranosyl)-5-(1H-pyrazol-5-yl)-7H-pyrrolo[2,3-d]pyrimidin-4-amine,
7-(2-deoxy-2-fluoro-2-methyl-5-triphospho-β-D-ribofuranosyl)-5-(1,3-oxazol-2-yl)-7H-pyrrolo[2,3-d]pyrimidin-4-amine,
7-(2-C-methyl-5-triphospho-β-D-ribofuranosyl)-5-pyrimidin-2-yl-7H-pyrrolo[2,3-d]pyrimidin-4-amine,
7-(2-deoxy-2-fluoro-2-methyl-5-triphospho-β-D-ribofuranosyl)-5-(1,2,4-oxadiazol-3-yl)-7H-pyrrolo[2,3-d]pyrimidin-4-amine,
7-(2-deoxy-2-fluoro-2-methyl-β-D-ribofuranosyl)-5-(1H-pyrazol-5-yl)-7H-pyrrolo[2,3-d]pyrimidin-4-amine,
7-(2-C-methyl-β-D-ribofuranosyl)-5-(6-methoxypyridin-3-yl)-7H-pyrrolo[2,3-d]pyrimidin-4-amine,
7-(2-deoxy-2-fluoro-2-methyl-β-D-ribofuranosyl)-5-(1,2,4-oxadiazol-3-yl)-7H-pyrrolo[2,3-d]pyrimidin-4-amine,
7-(2-deoxy-2-fluoro-2-methyl-β-D-ribofuranosyl)-5-(1,3-oxazol-2-yl)-7H-pyrrolo[2,3-d]pyrimidin-4-amine,
1-(2-C-methyl-β-D-ribofuranosyl)-3-(1H-pyrazol-3-yl)-1H-pyrazolo[3,4-d]pyrimidin-4-amine,
1-[5-O-(hydroxy{[hydroxy(phosphonooxy)phosphoryl]oxy}phosphoryl)-2-C-methyl- -D-ribofuranosyl]-3-(1H-pyrazol-3-yl)-1H-pyrazolo[3,4-d]pyrimidin-4-amine,
7-(2-C-methyl-β-D-ribofuranosyl)-5-(1,3,4-oxadiazol-2-yl)-7H-pyrrolo[2,3-d]pyrimidin-4-amine,
7-(2-C-methyl-5-triphospho-β-D-ribofuranosyl)-5-(1,3,4-oxadiazol-2-yl)-7H-pyrrolo[2,3-d]pyrimidin-4-amine,
Ethyl 2-{[(R)-({(2R,3S,4R,5R)-5-[4-amino-5-(1H-pyrazol-5-yl)-7H-pyrrolo[2,3-d]pyrimidin-7-yl]-3,4-dihydroxy-4-methyltetrahydro furan-2-yl}methoxy)(phenoxy)phosphoryl]amino}propanoate,
Ethyl 2-{[(R)-({(2R,3S,4R,5R)-5-[4-amino-5-(1,3-oxazol-2-yl)-7H-pyrrolo[2,3-d]pyrimidin-7-yl]-3,4-dihydroxy-4-methyltetrahydro furan-2-yl}methoxy)(phenoxy)phosphoryl]amino}propanoate
and pharmaceutically acceptable salts thereof.

10. A pharmaceutical composition comprising a compound according to claim 1 together with a pharmaceutically acceptable carrier.

11-13. (canceled)

14. A method of inhibiting HCV NS5B polymerase, inhibiting HCV replication, or treating HCV infection with an effective amount of a compound according to claim 1.

15. (canceled)
